(19) United States
(12) Patent Application Publication
(10) Pub. No.: US 2024/0000368 A1
(43) Pub. Date: Jan. 4, 2024

(54) SMELL SENSING SYSTEM

(71) Applicant: OWIDGETS LTD., Cambridge (GB)

(72) Inventors: Emanuela MAGGIONI, Cambridge (GB); Marianna OBRIST, Cambridge (GB); Richard HOPPER, Cambridge (GB); Florin UDREA, Cambridge (GB)

(21) Appl. No.: 18/249,841

(22) PCT Filed: Oct. 22, 2021
(86) PCT No.: PCT/GB2021/052754

§ 371 Date: Apr. 20, 2023

(30) Foreign Application Priority Data
Oct. 22, 2020 (GB) 2016810.0
Jul. 30, 2021 (GB) 2111060.6

(51) Int. Cl. A61B 5/00

(52) U.S. Cl. CPC A61B 5/4011; A61B 5/7475; A61B 2560/0252; A61B 2560/0257; A61B 2562/029

(57) ABSTRACT

The present application relates to a smell sensing system (100) comprising a smell delivery device (104) for delivering an olfactory output (110). The smell delivery device (104) comprises a delivery channel (3) for receiving a substance (5a) from a canister (5), the substance (5a) configured to produce an olfactory output (114). The smell delivery device (104) also comprises an output component (7) through which the substance (5a) is emitted. The smell delivery device (104) also comprises one or more airflow generating elements (13) configured to generate airflow to transport the substance (5a) from the canister (5) to the output component (7). The smell sensing system (100) also comprises a smell sensing device (102) for detecting the olfactory output (110) delivered by the smell delivery device (104). The smell sensing device (102) comprises at least one gas sensor (31, 32) configured to, in response to the olfactory output (110), generate sensor information (114) corresponding to the olfactory output (110). The smell sensing device (102) is configured to output the sensor information (114) to a processor (106).

### FIELD OF THE DISCLOSURE

The present disclosure provides a smell sensing system and associated methods. The present disclosure also provides a smell aiding system and associated methods. The present disclosure also provides an adaptive smell delivery device, system unit and method. The disclosure relates to a smell delivery device and an independent system unit that adapts its delivery specifications to varying environments and user interactions, for applications ranging from smell training and smell testing, for maintaining and measuring cognitive and olfactory capabilities, to education, entertainment, and intelligent and autonomous system use.

### BACKGROUND

Many people have impediments relating to their sense of smell. For some people this is a permanent condition, or symptom, whilst for others it is temporary. For example, some people suffering with COVID-19 reported a loss of taste or smell. Others, mainly older people, are specifically affected by smell loss and smell dysfunctions. In already frail people (e.g. elderly; people with long-term conditions) a cascade of deficit can follow from ignoring smell care needs, through weight loss, sarcopenia, and worsening frailty to falls and injuries. It can also lead to depression and anxiety [Speth et al. Laryngoscope, 2020]. It is now firmly established that being able to reliably measure and monitor a person's smell perception and detect changes can help identify and diagnose early stage onsets of cognitive or motor symptoms (even a decade or more before), for degenerative conditions such as Dementia, including Alzheimer's and Parkinson's disease. It is useful to determine the severity and nature of the loss of sense of smell in a diagnosis. Moreover, during treatment it is advantageous to be able to measure any improvements in the sense of smell of a patient.

Other people are employed specifically for their perceived quality of sense of smell. For example, sommeliers typically have a good sense of smell. Therefore, it is advantageous to be able to characterise, train and measure the ability of an individual to smell.

In still further applications, entertainment systems may deploy smell stimuli to users. For example, a system connected to a virtual reality headset, or the headset itself may emit smells to the user at specific times in order that the experience be more immersive and accessible to the user. Therefore, for all the above applications smell stimuli delivery is advantageous.

Several systems for smell stimuli delivery (odorants, chemical substances) via the environment, or directly oriented to a user's nose or a user's head space have been proposed. Depending upon the methodologies for the smell delivery, smell delivery systems can be generally classified into two main categories:
(i) Carrier approach, the chemical substance is delivered via a flow, for example carried by a gas (air) or air vortex towards the user' nose head space. The chemical substances are delivered by a carrier gas, typically pressurized, from a chamber.
(ii) Transformational approach, the chemical substance is presented in an enclosed volume and diffused through material status modification of such chemical substances. The status modification is usually obtained via an atomiser (ultrasonic transducer or piezoelectric), jetting dispersion or evaporation through heating.

Examples of transformational approach smell delivery device can be found in different publications, as in U.S. Pat. No. 9,283,296 (“Scent producing apparatus”) by Yossi et al, which presents an apparatus of vibration plates with scent reservoirs to atomize to each scent contained in the reservoir. Further background can be found in system developed for assessing a person's sense of smell as in U.S. Pat. No. 6,557,394 (“Smell test device”) by Doty R. L., which presents a digital olfactometer that dispenses controlled amounts of a volatile test fluid from a digital jetting device though droplets dispensed onto a heater which vaporizes the fluid at a test location with head space resolution.

Transformational methods to delivery smell suffer from low accuracy in the temporal (slow) and spatial (nose and head space target with short distance coverage) control over the delivery, cross-contamination between channels and most importantly no real-time control over the smell intensity.

For exploiting olfaction (the sense of smell) in the field of Human-Computer Interaction and user interface applications, the carrier approach is the more suitable method, as it allows the manipulation of the technical parameters of the smell delivery (e.g., intensity, frequency, timing, position of the smell output/delivery) in the interaction between a user and a system/interface. Within the dynamic approaches the present common method is to generate an air flow with fans (as Exhalia Diffuser SBi4), vortex via air cannon (as in K. Murai, T. Serizawa and Y. Yanagida, “Localized scent presentation to a walking person by using scent projectors,” 2011 IEEE International Symposium on VR Innovation, 2011, pp. 67-70) or via external pressured air supplier (e.g., canister or compressor) (as in Schriever, V. A., Körner, J., Beyer, R., Viana, S., & Seo, H. S. (2011). A computer-controlled olfactometer for a self-administered odor identification test. European archives of oto-rhino-laryngology, 268(9), 1293-1297).

Different delivery methods and systems have been used to meet the specific requirements of the applications they have been developed for. In WO 96/37248 (“Brain-training method and device based on olfactory stimuli”), by J. Hessabi, presents a fan-based system to emit un-mixed smells into the room, specifically with a setup for training the brain to counteract age-related impairment, the delivery performance has low controllability over temporal and spatial smell diffusion. Similarly, in U.S. Ser. No. 10/610,147B2 (“Neurodegenerative disease screening using an olfactometer”) by M. W. Albers, presents systems and methods for an olfactometer for delivering computer-controlled smell stimuli, receiving input from the user for an odor (smell) discrimination test based on a threshold calculation for neurodegenerative screening. The described olfactometer referred to a commercially available portable device named OLFACT olfactometer produced by Osmic Enterprises of Cincinnati, Ohio. The olfactometer performs well in delivery accuracy in spatial and time resolution, with no cross-contamination between smell delivery channels, however, it suffers from a lack of real-time air-flow adjustment in response to user interaction, or environmental factors and there is no quantification of the quantity of smell delivery and there is a lack of flexibility in the delivery output configurations.

EP3142096B1 (“Olfactory Display”), by D. Wook and K. Ando, presents an apparatus to emit fragrances, contained in a plurality of chambers in solid form fragrances, using a blower fan system generating an air flow and by using a piezoelectric device, within a range bounded in terms of time and space with the ambition of being used in synchronization with audio-visual contents. The presented apparatus suffers from cross-contamination between smell stimuli delivered, slow delivery speed with relative long linger time of the delivered smell stimuli in the surrounding environment and performs poorly in the synchronisation with audio-visual contents.

U.S. Ser. No. 10/188,767B2 (“Scent presentation method, scent presentation apparatus, and olfaction improving apparatus”), K. Okada, S. Kanzaki, S. Horiguchi, describes a method of presenting scent through pulse ejection based on the determined presentation condition for maintaining and improving olfactory capabilities. This system suffers from cross-contamination between channels of smell delivery with an inaccurate control over the delivery itself, therefore suffering of a lack of precise and reliable delivery for determining a user's smell detection threshold.

A review of the commercially available mechanisms of delivery for automotive applications and their performance and capabilities has been published by D. Dmitrenko, C. T. Vi, and M. Obrist (“A Comparison of Scent-Delivery Devices and Their Meaningful Use for In-Car Olfactory Interaction”, Automotive' UI Proceedings, ACM, 2016). Similarly, a review of delivery devices used for notification efficacy through olfactory information, has been published by E. Maggioni et al., “Smell-O-Message: Integration of Olfactory Notifications into a Messaging Application to Improve Users' Performance”. Proceedings of the 20th ACM International Conference on Multimodal Interaction (pp. 45-54).

The state-of-the-art is concerned with efficient and accurate smell delivery devices (so called olfactometer), in terms of controllability over the delivery parameters, are technologies that use pressurized air flow for carrying the chemical substances (odorants). Computer controlled olfactometers have been proposed as presented in U.S. Pat. No. 8,899,095 B, by Lundström, J. N. et al., including an air-actuated olfactometer MRI compatible with external compressor for air-supplier presenting a different material component with the same functionalities and carrier of pressured air coming from an external supplier. Similarly, in Lundström, J. N., et al., 2010. “Methods for building an inexpensive computer-controlled olfactometer for temporally-precise experiments”, International Journal of Psychophysiology, 78(2), pp. 179-189, and in Johnson, B. N. and Sobel, N., 2006 “Methods for building an olfactometer with known concentration outcomes”, Journal of neuroscience methods, 160(2), 231-245, a highly accurate computer-controlled olfactometer is presented. The last two citations present detailed instructions for construction of an air-dilution olfactometer for laboratory and research-based applications. The two apparatuses work by flowing a given carrier gas at a given flow rate through a given odorant in a canister controlling accurately spatial and temporal delivery, without any contamination between channels. The devices use activated pneumatic valves and a photo-ionization detector to measure the actual concentration of the odorant in the gas delivered. The external pressurized air supply allows setting of the desired flow velocity, temperature, humidity and pressure, however without having an adaptive automatic system to set the ideal delivery parameters with regards to the user's interaction and environmental factors.

Traditional olfactometers are known, as shown in the previous citations, for efforts made in the direction of high accuracy and stimulus quantification. However, an important drawback of the previously mentioned citations is that the olfactometers shown and more accurately smell delivery devices formed by electro-valves are confined to laboratory equipment types e.g. bulky, heavy, expensive, and not suitable as consumer or home care products, requiring an external source of compressed air (i.e., compressor or pressurized tanks) necessary for generating the air flow. Such cited apparatuses frequently require integration of a cooling system and have high electrical power demand, without allowing a usability of the device for different applications in a laboratory setting, or a possible usage of such of apparatus for a range of consumer human-interface applications. Thus, there is a growing demand for highly accurate and controllable smell delivery systems, with high spatial and temporal resolution, with real time control over the smell delivery and adaptive to varying users' interactions and to different environments, in a wide range of consumer/medical or home care applications.

The current generation of smell delivery devices have, amongst others, the following limitations:
(I) Application-specific design, with limited generalisation and usability.
(II) Limited control and adaptability over smell stimuli specifications in real-time, with high operational latency.
(III) Fixed number of chemical substances containers and delivery channels, without a modular design approach.
(IV) Limited smell delivery device usage configurations, without a standardized user' data recording intra applications.
(V) Fixed smell output lengths and positions.
(VI) Trade-off between accurate and reliable smell delivery in high-end products (olfactometers) and size (bulky).
(VII) When in the category of high accurate and reliable devices, they frequently require an external unit supplying pressurized air to generate air flow.

The smell delivery device and method of the present disclosure are advantageous over the state-of-the-art devices for at least the following reasons:
(I) High-control over the delivery of the chemical substances, spatial and temporal diffusion accuracy with real-time feedback control.
(II) Adaptability to perceptual variabilities across users, users' interaction, different chemical substances, and environmental factors.
(III) Adaptable to a variety of usage applications, including control and configurability via a cloud-computing platform.
(IV) Miniaturized single-unit and modular smell delivery device design, with an internal unit to generate a breathable odourless air flow, without any contamination between channels.
(V) Modular and interchangeable containers for each chemical substance, without any contamination between containers.
(VI) Low operational latency (100-200 ms).
(VII) Ability to keep user's digital records accessible via a cloud-computing infrastructure or local data storage, interchangeable for different applications.
(VIII) Variety of options for system connectivity and configurability.
(IX) Flexible smell output lengths and configuration solutions, depending on the users' position, nose, or head space.

The present invention provides a smell delivery device that could be used as an autonomous device or could be incorporated within an adaptive smell delivery system unit. If used as part of a system, it could feature a cloud-computing configuration, for precise adaptive administration of smell stimuli or mixtures of such smell stimuli, with low latency, no-cross contamination between stimuli and real-time control to realise accurate, reliable, external stimulation to a person's nose or head space. According to this invention, the adaptive smell delivery device or the smell delivery system unit is capable of optimising and measuring the reproducibility of chemical substances, without interference with the structure of the chemicals, through the flow rate parameters, users' perceptual variabilities, user's biofeedback e.g. heartrate, skin conductance etc.), and environmental factors.

The presently disclosed system unit allows for synchronisation of the smell delivery with multi-delivery devices, across platform, enables digital records, and personalised user profiles.

The kind of highly accurate and adaptable smell delivery device and system described in the present innovation is fundamental for instance in case of healthcare applications in light of increased demands for personalised and precision medicine and advances in the digitization of health information, and may create new avenues for a range of consumer applications.

Smell sensing devices (smell sensors), often referred to as electronic noses, are conventionally intended to detect odours or flavours and to some extent they refer to the capability of reproducing human senses. Different techniques have been used to improve the sensitivity and selectivity of electronic noses. Sensor arrays based on polymer or metal oxide sensors have been employed. Algorithms using pattern recognition software based on neural networks or other machine learning techniques have also been applied.

In general, gas sensors and electronic noses have been used for identifying hazardous or toxic gases, for example ambient indoor or outdoor air quality, or for reproducing or discriminating between different olfactory sensations of different sources of smell, for example foods (different types of coffees, fresh milk, etc.). Other applications can be found in the beverage industry, agriculture and forestry, health-care, medical and security systems. A recent review of electronic noses and their applications can be found in Karakaya, D., Ulucan, O. & Turkan, M. “Electronic Nose and Its Applications: A Survey”. Int. J. Autom. Comput. 17, 179-209 (2020).

Most gas sensors and electronic noses are based on solid state technologies such as MEMS (Micro-Electro-Mechanical-Systems) or and/or CMOS (Complementary Metal Oxide Semiconductor) or CMOS compatible platforms. A recent review of such technologies can be found in Gary W. Hunter et al 2020 J. Electrochem. Soc. 167 037570.

The present disclosure aims at addressing one or more of the above mentioned problems.

### STATEMENTS OF INVENTION

Aspects of the invention are set out in the independent claims, optional features are set out in the dependent claims.

According to a first aspect there is disclosed a smell delivery device comprising:
a delivery channel for receiving a substance from a canister, the substance configured to produce an olfactory output, such as a smell;
an output component through which the substance is emitted; and one or more airflow generating elements configured to generate airflow to transport the substance from the canister to the output component;
a flow controller for controlling the flow rate, or concentration, of the substance through the delivery channel to the output component;
wherein the flow controller is configured to control the flow rate, or concentration of the substance from the delivery channel to the output component in response to feedback from at least one of:
a sensor configured to sense the flow rate, or concentration of the substance, through the delivery channel; and/or
an environmental sensor configured to sense environmental conditions; and/or
a user feedback device configured to receive an input from a user.

It is noted that each of the sensor configured to provide direct or indirect indication of the flow rate through the delivery channel, the environmental sensor, and the user feedback device, all individually improve the accuracy and precision of the device. For example, the sensor configured to sense the flow rate through the delivery channel may be used to determine flow rate, or concentration within the delivery channel, and this information can then be used in a feedback control system to determine whether the more or less of the substance should be released. The environmental sensor is advantageous as it allows the output of the smell delivery device to be changed dependent upon the environmental conditions. Environmental conditions affect how smell is perceived (for example, just before a thunderstorm the air changes slightly, and so there is an appreciable difference to how smells may be sensed). By measuring the environmental conditions the smell delivery device can then change the flow rate/concentration of the first substance through the delivery channel. This means that change in the user's perception of the smell can be compensated for. This enables any measurement of the user's perception to be a fair test in all conditions. The user feedback device may enable the user to indicate their perception. This can be used to adjust the output of the smell delivery device, in order to make a precisely controllable test. Moreover, these three sensors may be used advantageously in combination with one another. For example, the user feedback device and the environmental sensor may be particularly symbiotic in nature. The user feedback may indicate that a user is particularly affected by the environmental conditions, and therefore any corrections based on the environmental sensor measurement(s) may be amplified. When all three sensors are used the device may be particularly accurate, precise and controllable.

Optionally, the smell delivery device may further comprise the canister, wherein the canister is configured to store the substance.

Optionally, the feedback device receives input from the user indicating:
whether the olfactory output is at a level the user can/cannot sense; and/or
the emotional response of the user to the olfactory output; and/or
the duration, and/or the intensity of the user's perception/sensation of the olfactory output; and/or
a comparison between different olfactory outputs. This feedback may be particularly useful when determining any changes to the flow rate/concentration of the first substance in the delivery channel. For example, if the purpose of a measurement is to determine the minimum level that an individual can smell finding the concentration/flow rate at which the user begins to smell is advantageous. The concentration/flow rate may be increased until a user reaches this point.

Optionally, when the feedback device receives input from the user indicating that the user cannot sense the olfactory output the flow rate is modulated by the flow controller.

Optionally, the environmental sensor is configured to sense at least one of: the temperature of the environment, the humidity of the environment, the pressure of the environment, the amount of/identify of pollutants present in the environment. Each of these may be advantageous. For example, temperature and humidity are well-known to affect the smell of a substance, as may pressure and the quantity/identity of pollutants present. Measuring these parameters allows the modulation of the flow rate/concentration of the first substance.

Optionally, the humidity of the environment or at a location within the delivery device is above a predefined threshold;
the temperature of the environment or at a location within the delivery device is above a predefined threshold; and/or
the pressure of the environment or at a location within the delivery device is above a predefined threshold;
   the flow controller is configured to modulate the flow rate of the substance.

Optionally, if the amount of pollutants is above a certain threshold the smell delivery device stops the flow of the substance, as the user's perception will be altered by the environment. This may be particularly advantageous if it is determined the pollutants are too concentrated to fairly test a user's perception of smell. For example, if there is a forest or moorland fire within a set distance of a location the air may smell of burning, which may overpower the smells emitted by the smell delivery device.

Optionally, the flow rate sensor is configured to communicate the flow rate measurement to the flow controller, and when the measured rate is different than the intended flow rate the flow rate is modulated until the intended flow rate is reached and stabilised. This may be advantageous as it may ensure the accuracy and precision of the smell delivery device. In particular, this may ensure that the flow rate/concentration of the substance does not deviate from a set amount.

Optionally, the sensor configured to sense the flow rate, or concentration (which may be determined directly or indirectly), is any of
a flow sensor, a pressure sensor, or a differential pressure sensor; and
wherein the airflow generating element comprises one or more pumps or micro-pumps; and
the flow controller comprises one or more of: adjustable valves, filters, electronic devices, circuits, memory devices, microcontrollers or microprocessors.

Optionally, the smell delivery device further comprises a distance sensor to determine the distance of the output component to the location of the user, and as the determined distance increases the flow controller is configured to modulate the flow rate, preferably the distance sensor is one of an optical distance sensor, an ultrasonic distance sensor or a CO2 sensor. The further a user is from the device the more spread out the substance emitted by the smell delivery device is (due to the larger volume of air between the user and the device). Therefore when the user is further away monitoring the distance of the user enables this variable to be accounted for. This means that the smell delivery device can be simpler, and more efficient, as it is not necessary to control as many variables as previously.

Optionally, the smell delivery device further comprises:
a second delivery channel for receiving a second substance from a second canister, the second substance configure to produce a second olfactory output, or alter the olfactory output associated with the first substance;
a second output component through which the second substance is emitted; and
wherein the flow controller is configured to control the flow rate of the second substance from the second delivery channel to the second output component in response to feedback from at least one of:
a second sensor positioned in the second delivery channel configured to sense the flow rate through the second delivery channel; and/or
the environmental sensor configured to sense environmental conditions; and/or
the user feedback device configured to receive an input from a user,
preferably further comprising the second canister configured to store the second substance; and
preferably wherein the flow controller comprises an array of flow controllers, wherein each flow controller in the array is configured to control the flow rate, or concentration, of the second substance, from the second delivery channel to the second output component. Emitting a second substance is particularly advantageous. It may allow smells to be mixed to form a new smell, or to change the strength of the smell caused by the first substance. It allows multiple smells to be tested with a single device. The second delivery channel may referred to as one or more further delivery channels.

Optionally, the smell delivery device further comprises a smell output extension forming a channel into which the first and second outputs feed, and a single user output at the distal end; and/or that the first substance and the second substance mix to form a mixture with an olfactory output different to the olfactory output of the first substance, and the olfactory output of the second substance.

Optionally, the first delivery channel and the second delivery channel are isolated from one another to avoid contamination of the first and second delivery channels. This is particularly advantageous to preserve the integrity of each channel through multiple uses so that each use of the smell delivery device emits the intended smell.

Optionally, the smell delivery device further comprises a noise generating element configured to generate noise sufficient to make the operation of the smell delivery device unnoticeable, so that the user is not aware whether the device is in use. Users may otherwise notice when the smell delivery device is emitting a substance based on a noise produced by the flow controller. From this they may be able to claim (or may even believe) they have sensed the smell produced, when this is not the case. This therefore creates a fair test for when the smell delivery device is in use, for example in a clinical setting.

Optionally, the smell delivery device further comprises one or more airflow generating elements configured to generate airflow to transport the substance from the canister to the output component. This may allow the substance to be directed to the user faster than relying on a diffusive process.

Optionally, the airflow is configured to be odourless so that the smell of the substance is unaltered, preferably the smell delivery comprises an air inlet element to take air from the environment and one or more air filters to remove pollutants from the air.

Optionally, the smell delivery device further comprises a communication unit configured to receive instructions from a second device. This enables the smell delivery device to be controlled externally, for example by a physician.

According to a second aspect there is disclosed a canister comprising a storage volume storing a substance, wherein the substance is associated with an olfactory output, wherein the canister is configured to be received by the smell delivery device of the first aspect, and wherein once received within the smell delivery device, the canister is configured to emit the substance into the delivery channel. The removable nature of the canister may be advantageous as it may allow the smell delivery device to be replenished once the substance originally in the canisters has been used. This may also allow maintenance of the device to be performed and ensures that a larger range of smells may be tested by the device.

According to a third aspect there is disclosed an adaptive system unit comprising: an input unit and a communication unit, the input unit configured to receive an input from a user, and the communication unit configured to communicate with the smell delivery device described above, wherein the communication with the smell delivery device comprises instructions to affect the olfactory output emitted by the smell delivery device, preferably the input unit is one of: a button, a screen, or is a detection of a user movement, such as the movement of a user's eyes. The adaptive system unit advantageously may communicate with the smell delivery device described above to control the output of the smell delivery device.

According to a fourth aspect there is disclosed system comprising the adaptive systems unit of the third aspect, and the smell delivery device of the first aspect.

According to a fifth aspect there is disclosed a method of delivering smell to a user from the smell delivery device of the first aspect, the method comprising the steps of:
receiving instructions to emit a flow of a first substance, wherein the substance has an olfactory output, such as a smell, associated therewith;
beginning the flow of the first substance at a first flow rate, or concentration;
receiving a measurement from one or more of:
the sensor positioned in the first delivery channel configured to sense the flow rate, or concentration, through the delivery channel; and/or
the environmental sensor configured to sense environmental conditions; and/or
the user feedback device configured to receive an input from a user; and
in response to the measurement changing the flow rate, or concentration, of the first substance. Changing the flow rate or concentration of the substance in response to the measurement from one of the three sensors may be advantageous as it enables the flow rate/concentration of the substance to be controlled accurately.

Optionally, the measurement is from the user feedback device and comprises an indication of at least one of:
whether the user can sense the olfactory output;
the duration of the user's perception of the olfactory output;
the user's emotional response to the olfactory output. This information may be useful in determining the flow rate/concentration of the first substance that should be used in the remainder of a test.

Optionally, the measurement is from the environmental sensor and comprises at least one:
an indication of whether the humidity of the environment is above a predefined threshold;
an indication of whether the temperature of the environment is above a predefined threshold; and/or
an indication of whether the pressure of the environment is above a predefined threshold. Each of these may be advantageous. For example, temperature and humidity are well-known to effect the smell of a substance, as may pressure (which is also linked with rainfall). Measuring these parameters allows the modulation of the flow rate/concentration of the first substance.

Optionally, the measurement is from the sensor positioned in the first delivery channel and comprises an indication that the measured flow rate is different to the intended flow rate, and in response the flow rate is modulated until the intended flow rate is reached and stabilised. This may be advantageous as it may ensure the accuracy and precision of the smell delivery device. In particular this may ensure that the flow rate/concentration of the substance does not deviate from a set amount.

Optionally, the measurement comprises an indication of the distance of the user from the device, and when the user is further away the flow rate, or concentration, of the first substance is increased. The further a user is from the device the more spread out the substance emitted by the smell delivery device is (due to the larger volume of air between the user and the device). Therefore, when the user is further away monitoring the distance of the user enables this variable to be accounted for. This means that the smell delivery device to be simpler, and more efficient, as it is not necessary to control as many variables as previously.

According to a sixth aspect there is disclosed a computer program product comprising program instructions configured to perform the method of the fifth aspect.

According to a seventh aspect there is disclosed a programmable device, programmed with the computer program product of the sixth aspect, for example the programmable device is a mobile phone, tablet, laptop, or desktop computer.

According to an eighth aspect there is disclosed a smell aiding system for aiding and replacing the sense of smell of a user, comprising:
a smell sensing device for detecting an olfactory output, comprising:
   at least one gas sensor configured to, in response to the olfactory output, generate sensor information corresponding to the olfactory output;
   wherein the smell sensing device is configured to output the sensor information to a processor;
   and wherein the smell sensing device is attachable to a user to be arranged in proximity to the nose of the user; and
a user output device configured to receive instructions from the processor, and wherein the user output device is configured to, in response to receiving the instructions, output an identification of a smell associated with the olfactory output to the user.

By outputting an identification of a smell associated with an olfactory output, the system can aid or even replace the sense of smell of a user. For instance, where the sense of smell of a user is impaired (fully or partially impaired), an identification of the smell can instead be outputted via a different platform. The gas sensor (or electronic noses) can detect the olfactory output and in response generate sensor information. This may indicate information about the olfactory output and can be used to identify the smell. As the smell sensing device is attachable to the user, the device can be placed in proximity to the nose of the user. In some examples, the smell sensing device is wearable. This preferably means that the smell sensing device can be worn by the user. The smell sensing device can be positioned on, for example, glasses, necklace, or clothes of the user. The configuration can be on the nose or may be in the user olfactory perception headspace within where they can detect smells. This means that the measurement of the gas sensor can be very accurate and can be indicative of the olfactory output actually received by the nose of the user. This can be more accurate than providing the gas sensor at a distal location from the user.

Therefore, the attachment of the smell sensing device to the user allows the smell aiding system to aid or replace the sense of smell. Because the smell sensing device is in a location to mimic the detection of smell by the user's nose, the detection by the smell sensing device can be representative of the user's sense of smell. In cases where the user's sense of smell is impaired, the smell sensing device can accurately aid or replace the user's sense of smell. For example, the user output device can identify the presence or strength of a smell detected by the smell sensing device. Because the device can be located in the vicinity of the user's nose, this can correspond accurately to what the user would have perceived if they had a fully functioning sense of smell. Accordingly, the smell aiding device can be used to more accurately represent the user's sense of smell than, for example, being arranged further away from the user's nose such as by being distanced from the user's nose or even being hand-held. Furthermore, by being attachable to the user, sensing smell aiding device can be used hands-free, improving ease of operation and convenience.

The smell sensing device is attachable to the user, and may be attached and left in place. For example, the smell sensing device may comprise an attachment means for attaching the smell sensing device to the user. For instance, the attachment means may comprise a clip, which in some cases may clip onto the user's face or preferably their nose. In some examples, the attachment means may be adjustable to secure the smell sensing device is position. For instance, the attachment means may be an adjustable clip, which in some cases can be adjusted to fit over and secure on the user's nose.

The smell sensing device may be attachable to the user near the face of the user. In particular, the smell sensing device may be attachable to the user's face. Preferably, the smell sensing device may be attachable to the user's nose. The smell sensing device may be attachable to the user such that the gas sensor is located near the user's nose, for example when attached the gas sensor may be located less than 10 cm from the user's nose. In this case, this may refer to any part of the user's nose, and preferably to the user's nostrils.

As used herein, the olfactory output should preferably be understood to be an odour, for example from an odorant. The olfactory output, or odour, may be detectable by a human through their sense of smell. In particular, the olfactory output should be detectable by a human through their sense of smell within the norms of the human smell capability and assuming the sense of smell is not impaired. The olfactory output may otherwise be referred to as an odour. The olfactory output may otherwise be referred to as a smell. For example, the olfactory output may comprise volatile organic compound (VOC) chemicals which result in a smell. The output of the gas sensor may be indicative of the smell of the chemicals of the olfactory output. For example, the olfactory output may be considered as a smell when detected by a human nose (or other animal with an olfactory system). In some examples, the olfactory output may be from an object which emits an olfactory output. For example, lavender flowers may emit an olfactory output that results in a smell of lavender to a human. In some examples, the olfactory output may be provided by a smell delivery device such as disclosed herein.

In some cases, the instructions received by the user output device from the processor are based on the sensor information, as described in more detail below. For example, the processor may process the sensor information to generate the instructions so that the instructions to output the identification of the smell are based on the sensor information.

Preferably, the smell sensing device is arranged within 1 m of the nose of the user, more preferably within 50 cm, even more preferably within 10 cm, still more preferably within 2 cm. Preferably, proximity to the nose of the user refers to proximity to the nostrils of the nose of the user. Preferably, the smell sensing device is also detachable from the user.

The smell aiding system can therefore be used to provide the user with an output based on a detected olfactory output in proximity to the nose. In the event that the user has an impaired sense of smell, the output can assist or replace the sense of smell of the user. The output can identify the smell detected to supplement the sense of smell of the user or entirely replace it.

Optionally, the identification comprises a representation of an object associated with the smell. In some examples, the identification comprises a representation of the smell. For example, the representation may be a representation of an object that produces the smell. The representation may involve descriptors or triggers from other sensory modalities (e.g. audio, visual, tactile, gustatory). The representation may be using a different platform as described above. The platform can be descriptive in words, or visual in the form of images or videos or even audio. The platform could be integrated on a smart programmable device, such as a smartphone or tablet. For example, the smell of a particular flower can be represented by the picture of that flower displayed on the smart phone. The representation may be a visual representation such as a graphical representation (e.g. an image) and/or a text representation. For instance, the representation may be an image of lemons where the smell is a lemon smell. The identification of the smell may be output via a non-smell based platform. In other words, the identification may not be in smell form. For example (as described above), the identification may be vision-based, audio-based, and/or tactile-based. The representation may also be abstract. For example, a particular smell can be associated with a particular sound or an abstract image.

The processor can process the sensor information to identify the smell. This can involve pattern matching or other algorithms to correlate the sensor information with a smell. For example, the processor may access a database of correlations between sensor information and smells. For instance, if a smell results in a particular shape of the electrical output signal of the sensing device (e.g. shapes of the signal from individual sensors within the sensor array) in the sensor information, the shape of the signal can be analysed and matched with a particular smell or combination of smells. Machine learning (artificial intelligence platforms, such as neural networks) can be employed to perform this analysis. For example, a neural network platform can be used. A variety of controlled smells (with different intensities), and combinations of smells produced by a smell delivery device produce different shapes of the output signal from the sensor array of the sensing device. These can be used to train the neural network. In operation, when an unknown shape of an output signal is received, the neural network can identify the smell, or the combination of smells and their intensities.

The processor can select an identification of the olfactory output, for example selecting an image or text representing the smell. The processor may access a database storing the representations of the smell. The processor may then retrieve the representation of the identification and send it to the user output device. Otherwise, the processor may send instructions to cause the user output device to obtain the particular representation of the identification, for example from a database accessible by the user output device.

The identification of the smell may enable the user to understand what the smell is without being able to use their sense of smell. For example, where the user has an impaired sense of smell, the identification can allow the user to understand the smell even though their sense of smell does not permit this.

In some examples, the identification may also comprise an identification of a combination of smells. For example, the olfactory output detected may comprise a plurality of olfactory outputs mixed together. This may be intentional, for example during testing, or may be unintentional, such as in a natural environment where smells get mixed together. For example, the identification may identify one or more smells. In some examples, the identification may indicate an intensity of the one or more smells. The intensity may be a relative or absolute intensity, for example of each component smell. In some examples, the identification may identify a predominant smell, and may or may not identify smells which are less strong. This can be used for smell training, where a user such as a wine sommelier must be able to sense complex smells with different component smells. Even where there is a single smell, the identification may further identify an intensity of the smell. For example, this may be a relative or absolute intensity which can aid the user by informing them of how strong the odour is. For example, this could be used in aiding the user determining whether food is safe based on how strong an odour is, for example of milk.

In some examples, the smell aiding system is used for smell testing, smell training, and/or immersive experience. The smell sensing device in combination with the user output device allows the system to be used for testing a user ability to smell different olfactory outputs. The smell aiding system can also be used for smell training where the gas sensor can verify a user perception of smell. The smell aiding system can also be used for an immersive experience, such as in virtual reality (VR) or augmented reality (VR) entertainment, where the gas sensor can confirm smells emitted which can result in a user output such as a change in the visual or audio environment.

The user output device may be configured to output to the same user as the user using the system. For example, this may be the same user using the user input unit. The user output device may also output to an additional user such as a clinician performing a smell test on the user.

Optionally, the user output device comprises a visual output, and the identification comprises an image, text, and/or video outputted via the visual output. For example, the visual output may be a display or screen. The identification can be a representation of a smell associated with the olfactory output. For example, where the olfactory output detected by the gas sensor is determined to smell of lemons, the identification may comprise a picture of lemons, text of the word ‘lemons’, or a video of lemons. The intensity of the smell can also be represented in words or a diagram. In this manner, the visual output can supplement the sense of smell of the user and can be used to assist in cases where the user has difficulty smelling this olfactory output.

Optionally, the user output device comprises an audio output, and the identification comprises audio outputted via the audio output. For example, the audio output may be a speaker or other audio emitter. For example, where the olfactory output detected by the gas sensor is determined as a particular smell, the identification may comprise an audio clip associated with the smell, music associated with the smell, or a voice clip saying the smell. The user output device may comprise a visual output and an audio output to output a visual output and audio output separately or in combination.

Optionally, the user output device comprises a tactile output, and the identification comprises a tactile feedback outputted via the tactile output. For example, the tactile output may be a vibrator or other touch-based emitter. The tactile output may be attached to the user so that the user can sense the tactile output in response to the smell, for example by vibrating against the skin. The user output device may comprise a tactile output and a visual output and/or an audio output to output a tactile output and a visual output and/or an audio output separately or in combination.

In some examples, the user output devices comprises a gustatory output (i.e. relating to taste) or an olfactory output (i.e. relating to smell). In some cases, the user may have a partially impaired sense of smell, unable to detect the olfactory output, but the user output device can output the taste or smell at a higher level which can be detected by the user. The user output device can include a smell delivery device such as disclosed herein, in one example. This can be used to aid the smell of the user.

In some examples, the user output device is configured to, in response to receiving the instructions, output an indication of an intensity of the olfactory output. This can inform the user of a relative strength of the smell. For example, the user output device may output the identification as an image of lemons and can further supplement this with an indication of an intensity such as a number on a scale e.g. 1 to 10, which may be in the same form such as an image or text, or in a different form such as via audio.

Optionally, the sensor information comprises an indication of one or more of: presence of the olfactory output; an intensity of the olfactory output; an identification of the smell or a type of smell associated with the olfactory output; a pulse duration of the olfactory output; a duration between subsequent pulses of the olfactory output; a base line of the olfactory output; and/or whether the olfactory output is static or dynamic. For example, the sensor information may contain information that confirms whether or not an olfactory output has been detected or not, such as by comparison to a base line representing a background olfactory output. The sensor information may identify an intensity of the olfactory output, for example expressed as a voltage which may be converted to relevant intensity units. This can represent a relative strength or concentration of different olfactory outputs. The sensor information may identify the smell or type of smell where such processing is available on the smell sensing device. The sensor information may contain a pulse duration which is preferably a time for which the olfactory output was detected until the intensity falls back to the base line, or a duration between subsequent pulses. The sensor information may contain information related to the base line which may be a background smell over time. This can be used to isolate pulses of olfactory outputs and remove background effects. The base line may also indicate environmental olfactory outputs and olfactory outputs of the user. The sensor information may indicate whether the olfactory output is static or dynamic, which is preferably how the olfactory output changes over time or whether there are different smells over time. One or more of these may be provided in isolation or combination. The sensor information may also indicate smell selectivity. The sensor information may also contain synchronization signals or other control data.

Optionally, the smell aiding system further comprises the processor. In other words, the processor may be part of the smell aiding system. In other cases, the processor is independent, and the smell aiding system may be provided separately and used in combination with a processor.

Optionally, the processor is configured to process the sensor information to identify a smell associated with the olfactory output detected by the gas sensor, wherein the processor is configured to generate instructions corresponding to the identification, and wherein the processor is configured to provide the instructions to the user output device.

Optionally, the processor is configured to use machine learning, neural networks, and/or artificial intelligence algorithms to process the sensor information to identify the smell. For example, models can be trained using input and output data in order to improve accuracy. Many different olfactory outputs and intensities may be provided, and the resulting sensor information may be used to train the models. For a given output (i.e. sensor information), the trained models can more accurately infer the input (i.e. the olfactory output). This can increase selectivity and sensitivity to the olfactory outputs.

Optionally, the processor is configured to receive user information, and the processor is configured to correlate the sensor information with the user information. For example, the correlation may be used in identifying the smell. The user information can be used in conjunction with the sensor information to make a more accurate determination. The user information can also be used to affect the output. For instance, if the user information identifies a relative level of smell ability of the user, the output may be affected. For example, if the user information identifies that the user can perceive a certain level of particular smells, the system may output the identification in a different manner (for example only outputting an image rather than a more invasive audio output), or may not output an identification if the determined intensity is above a threshold level above which it is known that the user can detect the olfactory output.

Optionally, the user information comprises an indication of one or more of the following, for a particular user: age; gender; other demographic information; health information; and/or historical sensor information. This demographic or health information may be used to infer characteristics on ability to smell, which may affect the output.

Optionally, the processor is configured to receive the user information from the smell sensing device, the user output device, and/or an external database. For example, the smell sensing device and/or the user output device may be configured to send user information to the processor, for instance having a storage comprising the user information. In other examples, the processor may access an external database which may contain user information.

Optionally, the smell aiding system is configured to collect user information relating to a user. For example, the user may input user information. This may then be stored as part of the personal user information for the user for later use. This may also be aggregated with user information from other users, in which case it may be anonymised. The aggregated user information can then be used as general user information providing data relating to groups of users selected by e.g. demographic information.

Optionally, the smell aiding system further comprises an environmental sensor configured to, in response to an environmental parameter, generate environmental information corresponding to the environmental parameter, and the environmental sensor is configured to output the environmental information to the processor. The environmental information can then be processed.

Optionally, the environmental parameter is one or more of: humidity; temperature; pressure; an identification or intensity of pollutants; a distance between the smell sensing device and the nose of the user; respiration of the user. Different environmental factors can affect a sense of smell, so by considering these, the accuracy can be improved. Humidity, temperature, pressure, and pollutants can each affect the background environment and the detected olfactory output in light of this. The position between the smell sensing device and the nose of the user can be used to determine how accurate the gas sensor detection is compared to that perceived by the user, where the closer the gas sensor the more accurate the measurement. In other words, the system may comprise a position sensor for detecting a distance between the smell sensing device and the nose of the user. The distance can also be used to compensate the sensor information, especially when the sensor is calibrated to determine a compensation for a given separation. The intensity perceived at the nose of the user can then be determined. The respiration of the user can also affect the detected olfactory output. The sensor may detect magnitude and direction of nasal flow, and timing of breathing. For example, magnitude and direction of nasal flow can affect how the olfactory output is measured. By compensating for this, the measurement can be more accurate. In other words, the system may comprise a respiration sensor for detecting respiration of the user. The environmental information may also be features of the gas sensor response to the olfactory output (chemical stimuli) which can be used to indicate the smell type. For example, the environmental sensor may be a temperature sensor and/or a humidity sensor.

Optionally, the environmental sensor comprises at least part of the smell sensing device. For example, the environmental sensor may be arranged on the smell sensing device. This allows the compensation to be more accurate as the environmental sensor can be closer to the nose of the user and the gas sensor so is a better representation of the environment at the nose of the user. For example, the smell sensing device may comprise one or more gas sensors as well as one or more environmental sensors. For example, the smell sensing device may comprise one or more gas sensors and a temperature and/or humidity sensor. The one or more gas sensors may be an array of gas sensors, such as metal oxide sensors or a combination of metal oxide sensors and a PID sensor. Different metal oxide sensors can sense different smells, while the temperature and humidity sensors can compensate for the distortions in the gas sensor signal due to changes in humidity and temperature.

Optionally, the processor is configured to receive the environmental information, and the processor is configured to correlate the sensor information with the environmental information. For example, the correlation may be used in identifying the environment smell (or background smell). The environmental parameters may affect the perception of smell, so by processing this information, the processor can more accurately identify the smell and compensate for any environmental effects. For example, where the humidity is of such a level to affect detection of an olfactory output, the sensor information can be compensated to take this into account.

In some examples, the processor is configured to correlate the sensor information with the environmental information and the user information. For example, the correlation may be used in identifying the smell or combination of smells or the predominant smell or the intensity of the smell. By combining these sets of information, the sensor information can be compensated based on user historical data or preferences and environmental conditions, resulting in a more accurate identification. The output may also be affected. For example, if the humidity is such that the detection of the olfactory output is affected, the user may be alerted via the user output device, or the output may be adjusted to emit an audio response to supplement a visual response for example. In some examples, the processor is configured to correlate one or more of: the sensor information, the environmental information, and/or the user information. In some examples, the processor is configured to correlate the environmental information with the user information.

Optionally, the smell aiding system further comprises a user feedback device comprising a user input unit configured to, in response to receiving a user input indicating a user perception of the olfactory output, generate user input information corresponding to the olfactory output, wherein the user feedback device is configured to output the user input information to the processor. The user input information can then be processed. This can add a further aid of how the user perceives the smell to validate the gas sensor or to affect the output of the identification of the smell to the user. For example, if the user input information indicates that the user can correctly smell the olfactory output, an output of the identification may not be necessary. In another example, an incorrect user input may be used to identify a problem with the sense of smell or be used to feed into the user information stored that the user cannot sense that particular smell at that particular level or under those particular environmental conditions when combined with output from an environmental sensor, and the user may additionally be alerted via the user output device.

Optionally, the user input comprises an indication of the user perception of one or more of the following: presence of the olfactory output; an intensity of the olfactory output; an identification of the smell or a type of smell associated with the olfactory output; a pulse duration of the olfactory output; a duration between subsequent pulses of the olfactory output; and/or whether the olfactory output is static or dynamic. The user input can, for example, indicate that the user can or cannot detect a smell, which could be in the form of a binary input. The user input could relate to an intensity which may be qualitive (e.g. high, medium, low), quantitative (e.g. 1 to 10), or relative (e.g. higher, lower, or the same as a previous smell). The user input may allow for the user to attempt to identify the smell or type of smell either outright or from a subset of potential smells. The user input may also identify generally whether it is a positive or negative smell, or whether they like or dislike the smell, which can be fed into their user information.

Optionally, the user input unit comprises one or more of: a button; a touch-screen; and/or a detector for detecting user response. For example, in a smell testing or calibration environment, the user may be provided with a user input unit to record their perception of different smells at different intensities. The user input unit may be a button that the user can use to indicate simple responses or selection from lists. The button may be a keyboard button or mouse click. The user input may be available through a touch-screen through a standalone device or a computing device such as a computer, smartphone, or tablet. Alternatively, the user input unit may comprise a detector which detects the response of the user, such as the movement of a user's eyes or biofeedback response such as heart rate or blood pressure. The user input unit may also comprise a microphone allowing user input by voice. The voice input can then be processed using voice recognition technology.

Optionally, the user feedback device comprises at least part of the user output device. In some examples, the user feedback device is arranged on the user output device. This allows the system to be consolidated. For example, the user output device may comprise a screen and/or speaker for outputting visual and/or audio output. The user output device may also comprise the user feedback device so that the user can input their response, for example having an input in the form of a touch-screen, button, such as a keyboard or mouse click. The same device can be used, for example a smart device such as a smartphone or tablet. The smartphone or tablet can then provide a touch-screen which provides user output such as through the screen and/or speaker, and can also allow user input via the touch-screen and/or microphone.

Optionally, the processor is configured to receive the user input information, and the processor is configured to correlate the sensor information with the user input information. For example, the correlation may be used in identifying the smell. The user input information can be used in combination with the sensor information to identify a smell more accurately. For example, the user perception of the smell can confirm the reading of the gas sensor. An incorrect user perception can also be used to adjust the output, for example by alerting the user that their sense of smell is potentially impaired. Information from the user feedback device can be synchronised, correlated, or evaluated against information provided by the smell sensing device. While the information provided by the smell sensing device can be in absolute or quantitative form (e.g. concentration level, smell intensity, pulse duration) and may be more accurate, the information provided by the user through the user feedback device may be relative or qualitative (e.g. low, medium, high level of smell, short or long duration, or stronger, weaker, or the same in terms of comparison). This is particularly useful in smell testing to assess the performance of the user's sense of smell and their ability to differentiate between smells or different intensities of certain smells, but also in smell training to monitor the performance over time or identify anomalies in the training procedures. In other words, the sensor information can be used to verify the user perception of smell by comparing to the user input information.

In some examples, the processor is configured to correlate one or more of: the sensor information, the environmental information, the user information, and/or the user input information. In some examples, the processor is configured to correlate the sensor information with the user information, the environmental information, and the user input information.

Other sensors may be used such as motion sensors or optical sensors. Other information from these sensors may be provided to the processor and may be processed in a similar manner to described above. Such information can also be correlated with other information such as sensor information, user information, environmental information, and/or user input information. This may be used in wellbeing, healthcare, or medical applications.

Optionally, the processor may be configured to generate a user smell profile. The processor may use one or more of the following in generating a user smell profile: sensor information; user information; environmental information; and/or user input information. A user smell profile can be built up based on the specific details of the user. For example, the user information such as demographic and health information can be input to build a profile of the user. Historical smell data can also be used, such as how the user has responded to certain smells, whether the user can detect certain smells, or how they prefer to receive an output. The user smell profile could also identify the ability of the user to identify/discern different type of smells and their relative intensity, or the impairment to certain chemical components of a specific smell. A smell can be defined by a variety of different chemicals, so by knowing the chemical components the possible receptor impairments of a user can be identified. The processor may also identify similar smells based on the chemical components that are determined to be likely that the user is unable to detect. The user profile can hold historical data and compared (e.g. in a statistical manner) with other user profiles, for example depending on the user age or possible ongoing neuro-degenerative diseases. Sensor information can also be used to provide a base line olfactory output for the user based on the smell output from a user or their background environment. Environmental information can also be used to identify typical parameters such as temperature and humidity which are an average of those for the user, as this may be dependent on how the user uses the system. For example, where the user primarily uses this in their house, the environmental parameters may be fairly static, meaning the user smell profile can be used to predict environmental conditions. Algorithms can be used to predict conditions such as environmental conditions or a base line which are based on historical data, which may be weighted based on more recent data or data which is tagged based on matching criteria such as date, time, or geo-location.

Optionally, the user smell profile is indicative of one or more of: olfactory outputs perceived by the user; relative intensity of olfactory outputs perceived by the user; relative duration of olfactory outputs perceived by the user; effect of environmental parameters on olfactory outputs perceived by the user; base line of olfactory outputs from the user. In some examples, the identification of the smell is based on the user smell profile. In some examples, the output of the identification is based on the user smell profile. The user smell profile may be used to compensate the sensor information, or affect the output of the identification of the smell. For example, where the detected olfactory output corresponds to a particular smell the user cannot detect well, the system may output an indication of the smell (e.g. an image) regardless of the intensity because it is known the user struggles with this particular smell or type of smell.

Optionally, the processor comprises at least part of a local computing device. In other words, the processor may form part of the local computing device. The processor may be integral with the local computing device. For example, the processor may be arranged on the local computing device. In other words, the local computing device may comprise the processor. For example, the processor may be located on a physical piece of hardware. The local computing device may be local to the user in the sense that it can be connected to one or more of the smell sensing device or user output device either directly or over a local network. The processor may optionally be referred to as a processing means, a processing unit, or a processing device.

Optionally, the user output device also comprises at least part of the local computing device. In other words, the user output device may form part of the local computing device. The local computing device can be the same local computing device that comprises the processor, but in other cases it may be separate. The user output device may be integral with the local computing device. For example, the user output device may be also arranged on the local computing device. In other words, the user output device may be located on the same local computing device as the processor. Thus, the system can be consolidated, and the same device can be used for user output and processing. The processor can then output the respective identification and does not need to transmit the instructions to a different entity.

In some examples, the local computing device is a computer, smartphone, or tablet, or other smart device. For example, the local computing device can be a smartphone which has a processor to perform the processing operations and a touch-screen interface for outputting a visual output. In cases where the local computing device is a smartphone and processing capacity may be limited, it may be more efficient to perform at least some of the processing at a remote location, such as in the cloud. In this case, the local computing device may offload some of the processing tasks to a remote processing device, sending instructions to the remote processing device regarding the processing, and receiving the results back for instructing the user output device to output the identification.

Optionally, the processor comprises at least part of a remote computing device. In other words, the processor may form part of the remote computing device. For example, the processor may be arranged on the remote computing device. For example, the remote computing device may be cloud-based. For example, the processor may not be located on a physical piece of hardware which is local to the user. Instead, the processor may be remote so that it can be connected via a remote network such as the internet. The smell sensing device and user output device may communicate with the processor via a remote connection. For example, one or more of the smell sensing device and the user output device may contain a network connection to communicate with the processor via a remote network such as the internet. Otherwise, the communication may be via an intermediate device which has such a network connection.

Optionally, the processor comprises a wireless receiver for wirelessly receiving the sensor information from the smell sensing device, and/or wherein the processor comprises a wireless transmitter for wirelessly transmitting the instructions to the user output device. For example, the processor may wirelessly communicate with the smell sensing device and the user output device, such as via Wi-Fi, mobile broadband, Bluetooth, Zigbee, or other wireless (e.g. RF) communication platform. Preferably, the processor communicates with the smell sensing device and the user output device via Zigbee or more preferably Bluetooth as this may permit smaller electronics. In turn, the processor may be connected to a network such as the internet via a wired or wireless connection. For example, the processor may be connected to the internet via a Wi-Fi connection, and then to the smell sensing device and user output device via a Bluetooth connection.

In some examples, the smell sensing device is configured to output the sensor information to the processor over a communication link. The communication link may be wired or wireless. For example, the smell sensing device may comprise a transmitter for transmitting the sensor information to the processor. Optionally, the smell sensing device comprises a wireless transmitter for wirelessly transmitting the sensor information to the processor. Optionally, the user output device comprises a wireless receiver for wirelessly receiving the instructions from the processor. A wireless transmitter is preferable as it avoids the need for wires, improves ease of use, allows for the physical separation of the smell sensing device from the processor, and avoids tangled wires. For example, the wireless transmitter/receiver may be Wi-Fi, mobile broadband, Bluetooth, Zigbee, or other wireless (e.g. RF) communication platform. The wireless transmitter may be used to transmit information wirelessly to a computing device such as a smart or programmable device. Additionally or alternatively, the smell sensing device and/or the user output device may connect to the computing device and/or the processor via a wired connection. Thus, the transmitter may transmit a signal to the processor over a wired connection to output the sensor information to the processor. For example, a cable may connect the smell sensing device to the processor. For example, the smell sensing device may have a wired interface such as a USB connector for connecting to the processor in the form of a computing device. The wired connection may be permanent or may be temporary, for example a wire may be plugged into the smell sensing device and/or the processor for transferring information. The smell sensing device may have a communication link with the user output device. For example, the transmitter of the smell sensing device may be configured to transmit and/or receive information (e.g. wirelessly or via wires) with the user output device. For instance, the smell sensing device may transmit sensor information to the user output device. The user output device can then transmit the sensor information to the processor for processing, or the processor may be on the user output device and the sensor information may be processed by the processor of the user output device. Accordingly, in this example, the output of the sensor information by the smell sensing device to the processor results in the sensor information being available at the user output device.

In some examples, the processor may be a remote device such as a server or cloud-based resource. In such cases, the smell sensing device may transmit the sensor information to the processor directly (e.g. wirelessly over the internet), or may transmit the sensor information indirectly via the user output device. For instance, the smell sensing device may transmit the sensor information to the user output device (e.g. via a wired connection or wirelessly, such as over Bluetooth. The user output device may then forward the sensor information to the processor (e.g. via a wired connection or wirelessly, such as over Wi-Fi). In this example, the sensor information is output by the smell sensing device to the processor via the user output device. In some examples, the smell sensing device can have a short-range transmitter such as a Bluetooth transmitter requiring low power to communicate with the user output device, which may be a nearby smartphone, instead of the smell sensing device requiring a longer-range transmitter such as Wi-Fi transmitter to independently connect to the internet. This can reduce the complexity and therefore size and weight of the smell sensing device, which is important where the smell sensing device is attachable to the user, such as being clipped to the user's nose. Battery life can also be improved, and costs can be reduced. In other examples, the smell sensing device may comprise a long-range transmitter such as a Wi-Fi transmitter or use other wireless or mobile technology such as 3G, 4G, 5G.

In some examples, the processor may comprise software. The software may be in the form of an application or program which is installable on a computing device. For example, the system may include a computing device such as a smart or programmable device (e.g. smartphone) having an application installed containing software for performing the processing functionality of the processor. The computing device may have a user interface for outputting information, such as identifying the smell or sensor information.

In some examples, the smell sensing device comprises one or more of: a sensing circuit; an amplifying circuit; a filtering circuit; a signal processing circuit; read-out electronics; a logic circuit; an application-specific integrated circuit, ASIC; and/or memory. The memory may be to locally store data such as the sensor information. The circuits may be to process data such as the sensor information. For example, the memory may be flash memory. The circuits may be useful in increasing the signal to noise ratio, allowing for more accurate reading of the olfactory output, identifying different olfactory outputs, and establishing a clean base line of the olfactory output.

Optionally, the smell delivery device is battery operated, comprises a battery accumulator, and/or is solar powered. Otherwise, the smell delivery device may be powered, such as to the mains or chargeable via a cable from the computing device. Preferably, the smell delivery device is portable. The battery or accumulator may be charged via a cable when connected to the computing device. Preferably, the smell delivery device is battery powered.

Optionally, the smell sensing device comprises a memory for storing the sensor information for subsequent output. In some examples, the user feedback device comprises a memory for storing the user input information for subsequent output. The memory can store the sensor information so that the sensor information can be transmitted in batches, for example either periodically or when connected to the processor. Otherwise, the sensor information may be transmitted live as it is generated.

In some examples, the smell sensing device may be assembled on a printed circuit board (PCB) and may contain several individual circuit blocks connected via tracks placed on the PCB. The information such as the sensor information could already be in a processed form using amplifying, filtering, or signal processing circuits, or ASICs incorporated in the smell sensing device. The information could be post-processed by software or hardware in the processor. It may contain a metal or plastic lid with holes to expose sensitive elements to ambient olfactory output or ambient environmental conditions (e.g. humidity). The smell sensing device may use state-of-the-art assembly techniques such as chip on board assembly, flip-chip, stack die assembly or wafer level packaging. The smell sensing device may alternatively be built on a lead frame inside a microelectronic metal/plastic/ceramic package with a moulding compound or metal lid for protection and holes implemented in the moulding compound or metal lid to expose sensitive elements to ambient olfactory output or ambient environmental conditions (e.g. humidity).

Optionally, the smell sensing device is directly attachable to a body part of the user. Optionally, the smell sensing device is directly attachable to the head of the user. Optionally, the smell sensing device is directly attachable to the nose of the user. In this manner, the smell sensing device can be attached to a part of the body so that it is preferably in contact with the body. For example, the smell sensing device may be clipped to the skin of the user. This can allow it to be more closely located to the user's nose. In some examples, the smell sensing device is indirectly attachable to a user via clothing or glasses of the user. In this way, the device can be attached to clothing or glasses to that it is in proximity to the user but can easily be attached and detached. In some examples, the smell sensing device is attachable to the head of the user. In some examples, the smell sensing device is attachable to the face of the user. For example, the smell sensing device may be attached to the face or hair of the user, which places the gas sensor in proximity to the user's nose. The device may be adjustable to finely tune the separation between the sensor and the nose. In some examples, the smell sensing device is attachable to the nose of the user. This ensures the gas sensor is as close to the nose of the user as possible, improving how representative the readings are of how the user's nose has received the olfactory output.

Optionally, the smell sensing device comprises a clip for attaching to the user or to clothing or glasses of the user. In some examples, the smell sensing device comprises a clip for attaching to the user or to clothing or glasses of the user. A clip can allow the device to easily be fitted and detached from the user as desired. The clip can attach directly to the user, such as to a body part. For example, the clip may attach the device to the nose of the user. Alternatively, the clip may attach to clothing so as to be minimally intrusive, or to existing glasses for convenience. The clip may attach to position the device in close proximity to the user's nose.

Optionally, the smell sensing device is indirectly attachable to a user via clothing or glasses of the user. Optionally, the smell sensing device is attached to a pair of glasses. For example, the system may include a pair of glasses that include the smell sensing device located thereon. The device may be permanently attached or secured to the glasses. The glasses may be spectacles or other eyewear such as goggles. The glasses may be dummy glasses without working lenses, or they may be tailored to the user, for example containing their prescription. This allows the user to wear glasses as usual, and avoids the need for clips. The gas sensor, any other sensors, processors, or transmitters can be incorporated into the structure of the glasses.

Optionally, the at least one gas sensor comprises at least one volatile organic compound, VOC, sensor. For example, the at least one gas sensor may comprise a plurality of gas sensors. In some examples, the at least one gas sensor may comprise a plurality of VOC sensors. Most smells are generated by volatile organic compounds, so a VOC sensor can be used to detect most olfactory outputs. Preferably, the sensor is based on CMOS or MEMS technologies. In other examples, the gas sensor may detect other gases, for example inorganic compounds. For example, the gas sensor may detect olfactory outputs such as those resulting from ammonia or hydrogen sulphide, which are not organic compounds but cause a smell.

In some examples, the at least one VOC sensor comprises at least one of: a photoionization, PID, gas sensor; a metal oxide, MOX, gas sensor; an electrochemical gas sensor; a polymer gas sensor; and/or a catalytic gas sensor.

The photoionization (PID) gas sensor may be operated in the ultraviolet (UV) range. The principle of operation is based on exposing the chemicals (VOC) which produce smell to high energy photons (typically from a UV source) which splits them into positive ions and electrons. This charge separation leads to a useful current which is then amplified. The greater the concentration of the chemicals (sub ppb to thousands of ppm levels) the higher the current produced. By measuring this current, the chemical concentration can be detected. The PID can produce live readings and can be operated continuously or in pulse mode. They are very stable and reliable. They are more expensive and slightly more bulky than other VOC sensors. PID sensors are not particularly selective and cover a wide range of VOCs. PID sensors can detect all VOCs that have ionization energies equal or lower than the energy of the photons emitted by the source. However, this also means that all VOCs with higher ionization energy than the source photon energy cannot be detected.

Metal oxide (MOX) sensors are based on a physical/chemical reaction at the surface (or close to the surface) of a sensing layer made of a metal oxide material, with a target gas (VOCs) at a specific temperature. The gas concentration is measured by monitoring the change in resistance of the sensing layer. The MOX sensor can operate at high temperatures and to reduce their power consumption they can be configured to operate in pulse mode. They can feature a heater (or micro-heater) suspended on a dielectric membrane (in MEMS or CMOS/MEMS technologies) to further reduce the power consumption and be able to operate at very high temperatures (typically between 200 to 500° C.). Different metal oxides can be employed such as tin oxide, tungsten oxide, zinc oxide, or alumina oxide or a combination. In addition, MOX layers can be doped with different materials such as platinum or palladium to control the electrical resistance and/or increase the sensitivity and/or stability to different gases. MOX arrays (monolithically integrated or in a discrete form) can be used to increase selectivity to different gases or different VOCs. MOX sensors are affected by temperature and humidity and a temperature and humidity sensor may be used to compensate for these effects. MOX sensors have high sensitivity and can be manufactured in high volume at relatively low cost. However, they are less stable than PID sensors and can drift in time and temperature. The base line can also be unstable. Algorithms can be implemented to improve the performance, selectivity, base line or self-calibration, for example using an ASIC or other type of hardware/software.

Electrochemical sensors are based on an electrochemical reaction that results in an electric current amplified and monitored through an external circuit. Electrochemical sensors feature a gas-permeable membrane and working (active) and counter (reference) electrodes. The magnitude of the current is given by how much of the target gas (VOCs) is oxidized or reduced. Electrochemical sensors have good linearity, and they have relatively good sensitivity. However, they are bulky and have a reduced lifetime.

Polymer sensors are similar in operation to MOX sensors but generally require lower temperature of operation. They can be made in a small form factor and can have lower power consumption. However, they tend to be less reproducible than MOX sensors and their stability in time and temperature can be less good. Nanomaterials could be employed to enhance surface properties of MOX or polymer sensors and/or improve sensitivity, selectivity, or stability.

Other types of gas sensors or VOC sensors can be based on catalytic techniques (e.g. pellistors), optical techniques (non-dispersive infrared (NDIR), photoacoustic). Catalytic sensors are based on a chemical reaction (such as oxygen burning) in the presence of a catalytic material at high temperatures. NDIR optical sensors are based on absorption of infrared radiation by the target gas.

In some examples, the at least one gas sensor comprises an array of gas sensors, wherein each gas sensor is configured to selectively sense different olfactory outputs with different intensities. One or more may be a VOC sensor. The array may be formed of discrete devices packaged individually or hybrid devices packaged together in the same housing or monolithically integrated within the same chip. Such sensors may contain micro-hotplates, membrane structures, and chemical sensing layers. In some examples, the at least one gas sensor comprises a first gas sensor and a second gas sensor. The first gas sensor may be a PID sensor. The second gas sensor may be a VOC sensor, preferably a MOX sensor.

Optionally, the at least one gas sensor comprises a first gas sensor and a second gas sensor, and the first gas sensor is configured to calibrate the second gas sensor. The first gas sensor can then be used to calibrate the second gas sensor. For example, known olfactory outputs and intensities can be initially used to calibrate the second gas sensor.

In some examples, both the first and second gas sensors are VOC sensors. Optionally, the first gas sensor comprises a PID sensor. This is particularly advantageous as the PID can be more accurate but often more expensive. In some examples, the second gas sensor comprises a MOX sensor which can be more cost effective. The PID sensor can be used to calibrate the MOX sensor or used to detect the smell of the user or the environment. The PID sensor could be used as a reference sensor detecting the total VOC, while the MOX sensor (or array of MOX sensors) can be used to distinguish between different chemicals. The MOX array may be integrated on-chip within one or more micro-hotplates, containing different types of metal oxide materials. Such metal oxide materials may be more sensitive to one VOC than another. By having an array of such MOX materials, it is possible, using classical or AI algorithms and read-out/processing circuit, to discern between different smells and their intensities.

The smell sensing device may be configured and calibrated to work with a smell delivery device such as disclosed herein. For example, the calibration may involve positioning the smell sensing device at a precise distance from the smell delivery device, calibrating the signals given by the smell sensing device against the delivery information (e.g. flow rate and intensity of the olfactory output) from the at least one channel of the smell delivery device. A calibrated smell sensing device or a reference smell sensing device may also be used to test or calibrate smell delivery devices and/or assess the quality and specifications of smell delivery devices in production. The smell sensing device could also be used as a means to indicate faults or misuse of the smell delivery device or to indicate whether the canisters of the smell delivery device are empty of chemicals or about to be empty.

Features of other aspects may be applied to this aspect. The smell aiding system of the eighth aspect may comprise any of the features of other aspects such as the first to seventh aspects, in particular the first aspect. In particular, the smell aiding system may further comprise the smell delivery device of the first aspect, and the smell aiding system can be used to provide an output to the user based on the olfactory output supplied by the smell delivery device. This can have particular application to smell testing, smell training, and immersive experience.

Disclosed herein is a smell sensing device for aiding or replacing the sense of smell of a user, comprising:
at least one gas sensor configured to, in response to detecting an olfactory output, generate sensor information corresponding to the olfactory output; and
a transmitter configured to output the sensor information to a processor;
wherein the smell sensing device is attachable to a user to be arranged in proximity to the nose of the user.

The smell sensing device may comprise one or more features of the smell sensing device of the smell aiding system disclosed herein. In some examples, the transmitter may be a wireless transmitter configured to wirelessly transmit the sensor information to the processor. In other examples, the transmitter may comprise a wired connection so that the smell sensing device is configured to output the sensor information to the processor over the wired connection. For example, the wired connection may be temporary such as by plugging in the smell sensing device to the processor periodically to download sensor information from the smell sensing device. In some examples, the processor may be located on the user output device disclosed herein. For example, the processor may be located on a computing device. The computing device may comprise a user output device. The user output device may be configured to output an identification of a smell associated with the olfactory output to the user. For example, in response to the user output device receiving the sensor information, the processor may process the sensor information. The processor may then instruct the user output device to output an identification of a smell in response to the processing of the sensor information, as described herein.

In other examples, the processor may be separate from the user output device. For example, the processor may be a remote device such as a server or cloud-based resource. In such cases, the smell sensing device may transmit the sensor information to the processor directly (e.g. wirelessly over the internet), or may transmit the sensor information indirectly via the user output device. For instance, the smell sensing device may transmit the sensor information to the user output device (e.g. via a wired connection or wirelessly, such as over Bluetooth™). The user output device may then forward the sensor information to the processor (e.g. via a wired connection or wirelessly, such as over Wi-Fi).

Disclosed herein is a user output device for aiding or replacing the sense of smell of a user, comprising:
a receiver configured to receive instructions from a processor, wherein the instructions are received from the processor in response to the processor receiving sensor information from a smell sensing device corresponding to a detected olfactory output;
wherein the user output device is configured to, in response to receiving the instructions from the processor, output an identification of a smell associated with the olfactory output to the user.

The user output device may comprise one or more features of the user output device of the smell aiding system disclosed herein. For example, the user output device may comprise a display to output a visual identification of the smell. The user output device can be used in conjunction with the smell sensing device above to aid or replace the sense of smell of a user. By detecting an olfactory output and outputting an identification of the smell to the user, the user's sense of smell can be supplemented or replaced by the indication. For instance, upon detection of a smell of lemons, an image of lemons can be displayed. This can help a user identify a smell if their sense of smell is impaired.

It will be appreciated that the smell sensing device and the user output device described above operate as interrelated products, in the sense that the two separate components are intended to interoperate with one another. In other words, the smell sensing device and the user output device, being intended to be used together, share a common underlying inventive concept.

According to a ninth aspect, there is disclosed a method of operating the smell aiding system of the eighth aspect, the method comprising:
generating, by the at least one gas sensor, sensor information in response to the olfactory output, the sensor information corresponding to the olfactory output;
outputting, by the smell sensing device, the sensor information to a processor; and
receiving, by the user output device, instructions from the processor based on the sensor information;
outputting, by the user output device, in response to receiving the instructions, an identification of a smell associated with the olfactory output to the user.

In some examples, the method further comprises performing operations associated with the smell aiding system of the eighth aspect. The identification of the smell may be as in the eighth aspect, such as further indicating an intensity of the smell. Features of other aspects may be applied to this aspect. Thus, any features disclosed in relation to the system of the eighth aspect may readily be applied to the method of the ninth aspect. In particular, the method may comprise outputting user information, environmental information, and/or user input information to the processor. The method may comprise providing the processor and the processor performing operations. For example, the method may comprise the processor receiving the sensor information, processing the sensor information to identify the smell, and optionally generating and providing the instructions to the user output device. The method may comprise the processor receiving the user information, environmental information, and/or user input information. The method may comprise the processor processing or correlating the sensor information with the user information, the environmental information, and/or the user input information. The method may comprise the processor generating and providing instructions to the user output device (for example identifying the smell), based on the processing or correlating.

According to a tenth aspect, there is disclosed a processing device for identifying a smell associated with an olfactory output, comprising:
a receiver configured to receive sensor information from a smell sensing device, the sensor information corresponding to an olfactory output detected by the smell sensing device;
a processor configured to process the sensor information to identify a smell associated with the olfactory output;
wherein the processor is configured to select a representation of the smell associated with the olfactory output, wherein the representation is a visual, audio, and/or tactile representation; and
a transmitter configured to send instructions to a user output device to output the representation of the identification of the smell associated with the olfactory output.

It will be appreciated that the processing device of the tenth aspect is complementary to the system of the eighth aspect. Features of other aspects may be applied to this aspect. The processing device may be further configured in a similar manner to the processor disclosed herein, and features of the processor of the eighth aspect may be readily applied to the processing device of the tenth aspect.

For example, the processor may be configured to correlate information as described above or use machine learning or other algorithms disclosed herein. The receiver and transmitter may be analogous to those disclosed herein, and may be the same component configured for receiving and transmitting or may be separate components. For example, the receiver and transmitter may be wireless for wirelessly communicating with the smell sensing device and the user output device.

According to an eleventh aspect, there is disclosed a method of identifying a smell associated with an olfactory output, the method comprising:
receiving sensor information from a smell sensing device, the sensor information corresponding to an olfactory output detected by the smell sensing device;
processing the sensor information to identify a smell associated with the olfactory output; and
sending instructions to a user output device to output the identification of the smell associated with the olfactory output.

Optionally, the method is computer implemented. The method may be performed by a processor such as the processor disclosed in the eighth aspect or the processing device of the tenth aspect. Features of other aspects may be applied to this aspect. Features of the eighth aspect may be readily applied to the method of the eleventh aspect. Method features of the processor can readily be applied to the eleventh aspect, such as receiving and processing other information such as user information, environmental information, user input information, and sending instructions based on such processing.

According to a twelfth aspect, there is disclosed a computer program, computer program product, or computer-readable medium comprising instructions which, when executed by a processor, cause the processor to perform the method of the eleventh aspect. Thus, the computer program may be in the form of software such as an application (or app). Features of other aspects may be applied to this aspect. Features of the eighth aspect may be readily applied to the twelfth aspect.

According to a thirteenth aspect, there is disclosed a computing device comprising the computer program, computer program product, or computer-readable medium of the twelfth aspect. For example, this may be a computer (e.g. desktop, laptop), a smartphone, a tablet, or other smart device (e.g. smart watch etc.) storing the computer program such as in the form of a software application. In another example, a computer-readable medium such as a computer data storage (e.g. hard drive) may be provided within a computing device. Features of other aspects may be applied to this aspect. Features of the eighth aspect may be readily applied to the thirteenth aspect.

According to a fourteenth aspect, there is disclosed a smell sensing system, comprising:
a smell delivery device for delivering an olfactory output, comprising:
   a delivery channel for receiving a substance from a canister, the substance configured to produce an olfactory output;
   an output component through which the substance is emitted; and
   one or more airflow generating elements configured to generate airflow to transport the substance from the canister to the output component; and
a smell sensing device for detecting the olfactory output delivered by the smell delivery device, comprising:
   at least one gas sensor configured to, in response to the olfactory output, generate sensor information corresponding to the olfactory output;
   wherein the smell sensing device is configured to output the sensor information to a processor.

The smell sensing system provides a smell delivery device and a smell sensing device for use in combination. The smell sensing device is configured to detect the olfactory output supplied by the smell delivery device. The sensor information can be outputted to a processor for processing the data from the gas sensor. This allows for validation of the smell delivery device, for example to verify that the delivery device is working correctly. The smell sensing device may be the same as the smell sensing device of the eighth aspect and may comprise one or more of the features described herein. The smell delivery device may be the same as the smell delivery device of the first aspect and may comprise one or more of the features described herein.

In some examples, the smell sensing system is used for smell testing, smell training, and/or immersive experience. By feeding back sensor information, the system can verify the smells supplied and use this to test or train a user's sense of smell. For example, the smell sensing device can generate sensor information which identifies the presence or strength of a smell, and this can be used to verify the correct functioning of the smell delivery device. As one example, if a canister containing a smell of lemons is inserted into the smell delivery device and the smell delivered, and the smell sensing device indicates a smell of lemons, then the operation of the smell delivery device can be confirmed. Otherwise, malfunction of the device may be indicated, or an empty canister may be identified. In another example, the smell sensing device can confirm the user's perception of smell by indicating the presence or strength of a smell to verify whether the user's perception of the smell was correct.

Optionally, the sensor information comprises an indication of one or more of: presence of the olfactory output; an intensity of the olfactory output; an identification of the smell or a type of smell associated with the olfactory output; a pulse duration of the olfactory output; a duration between subsequent pulses of the olfactory output; a base line of the olfactory output; and/or whether the olfactory output is static or dynamic. The sensor information may be the same as the sensor information of the eighth aspect. For example, the sensor information may contain information that confirms whether or not an olfactory output has been detected or not, such as by comparison to a base line representing a background olfactory output. The sensor information may identify an intensity of the olfactory output, for example expressed as a voltage which may be converted to relevant intensity units. This can represent a relative strength or concentration of different olfactory outputs. The sensor information may identify the smell or type of smell where such processing is available on the smell sensing device. The sensor information may contain a pulse duration which is preferably a time for which the olfactory output was detected until the intensity falls back to the base line, or a duration between subsequent pulses. The sensor information may contain information related to the base line which may be a background smell over time. This can be used to isolate pulses of olfactory outputs and remove background effects. The base line may also indicate environmental olfactory outputs and olfactory outputs of the user. The sensor information may indicate whether the olfactory output is static or dynamic, which is preferably how the olfactory output changes over time or whether there are different smells over time. One or more of these may be provided in isolation or combination. The sensor information may also indicate smell selectivity. The sensor information may also contain synchronization signals or other control data.

Optionally, the smell delivery device is configured to output delivery information to the processor corresponding to the substance emitted. The delivery information may provide information relating to the output of the olfactory output. This can be used to identify the smell or obtain other information about performance of the delivery device. For example, the delivery information may identify the canister or the smell of the canister for verification by the smell sensing device. This can be used to calibrate the smell sensing device by providing information of what the smell should be, which could be used to narrow down the potential matches of smell by the smell sensing device, or confirm a predicted smell. In other words, the delivery information can be used by the processor in determining the smell. Otherwise, this can be used to verify the accuracy of the smell sensing device by testing whether the results of the smell sensing device are accurate. In other words, the delivery information may not be used by the processor in determining the smell. In other examples, this can be used to verify the operation of the smell delivery device, because if no smell is detected but the delivery information indicates a smell has been released, then this could indicate malfunction of the smell delivery device.

Optionally, the delivery information is indicative of one or more of: the flow rate or concentration of the substance; pressure of a pump of the airflow generating elements of the smell delivery device; selection of delivery channels of the smell delivery device; selection or degree of opening of valves of the delivery channel of the smell delivery device; an identification of the smell or a type of smell; a pulse duration of the smell; a duration between subsequent pulses of the smell; a smell base line; and/or whether the smell is static or dynamic.

Optionally, the smell delivery device is configured to receive instructions from the processor, and wherein the smell delivery device is configured to, in response to receiving the instructions, adjust the delivery of the olfactory output. The instructions may cause the smell delivery device to adjust delivery of the olfactory output. For example, the instructions may cause the smell delivery device to cease emitting a substance due to a failure. The processor may determine a failure based on the sensor information received from the smell delivery device. The processor may determine a failure based on correlating the sensor information with the delivery information. For instance, if the delivery information indicates the delivery of an olfactory output, but the smell sensing device fails to register an olfactory output, the processor may determine a failure in the smell delivery device and/or the smell sensing device.

Optionally, the smell delivery device further comprises a flow controller for controlling the flow rate or concentration of the substance through the delivery channel to the output component. The flow controller may be the same as that described above in relation to the first aspect. For example, the flow controller may be a pump or fan to control airflow. This can be used to adjust the relative strength of the olfactory output. By providing different levels of flow rate or concentration, different combinations of smells can be produced, and the user's smell can be tested at different sensitivity levels.

Optionally, adjusting the delivery of the olfactory output comprises controlling the flow controller to adjust the flow rate or concentration. By controlling the flow controller to adjust the flow rate or concentration, the delivery of the olfactory output can be adjusted. For example, the higher the flow rate, the more VOCs carried with the airflow and delivered. Thus, the flow rate can be adjusted based on the sensor information from the smell sensing device. This permits the flow rate to be increased to increase the intensity of the olfactory output when the gas sensor detects a lower level than expected. This feedback ensures the olfactory output is accurate and as expected.

Optionally, the smell delivery device further comprises a plurality of delivery channels each for receiving a substance from a canister, the substance configured to produce an olfactory output. This allows multiple olfactory outputs to be delivered in quick succession or simultaneously, allowing combinations of smells to be generated. By providing dedicated delivery channels for receiving canisters, the system can be modular and easily separate smell sources from one another.

Optionally, adjusting the delivery of the olfactory output comprises selecting one or more delivery channels. For instance, the smell delivery device may select a particular delivery channel to cause emission of a substance from that delivery channel, while avoiding emission of a substance from a different delivery channel. This allows a particular olfactory output to be selected. In other examples, multiple delivery channels may be selected to varying degrees and mixtures of substances can be formed to generate a mixed olfactory output. For example, the smell delivery device may select a delivery channel not having a canister inserted (or having a canister containing air or odourless gas) to supply odourless air in conjunction with another canister supplying an olfactory product, with the result that the intensity of the olfactory product can be reduced by dilution in air, for example in response to sensor information indicating the intensity is too high.

Optionally, the smell delivery device further comprises a plurality of valves for controlling the plurality of delivery channels.

Optionally, adjusting the delivery of the olfactory output comprises opening or closing one or more valves or adjusting the degree of opening of one or more valves. The valves may be adjusted to control airflow through the channels and thereby select which substances are mixed to form the resulting olfactory output. This can allow precise control over the relative amounts of different olfactory outputs. Alternatively, the airflow speed can be adjusted for individual delivery channels to adjust the relative contribution.

Optionally, wherein the smell sensing system further comprises the processor. The processor may have one or more features described in relation to the processor of the eighth aspect. The processor may be arranged on the smell sensing device, on the smell delivery device, or as a further separate device.

Optionally, the processor is configured to correlate the sensor information with the delivery information to determine a validation of the delivery of the smell by the smell delivery device. The validation can be a simple check of whether the delivery is delivering an olfactory substance or not. This can be used to determine when the delivery device has completely failed or when the canisters are empty. The validation can also be more complex and involve a comparison of the intensity of the olfactory output compared to an expected level. In some examples, the processor may be configured to output the validation, for example by sending instructions relating to the validation to the user output device. For example, this may cause the user output device to output the validation, such as by indicating the validation by an error signal or alarm, e.g. a light.

Optionally, based on the validation, the processor is configured to determine an adjustment to the delivery of the olfactory output, wherein the processor is configured to generate instructions corresponding to the adjustment to the delivery of the olfactory output, and wherein the processor is configured to provide the instructions to the smell delivery device. The processor can determine an adjustment necessary to restore the delivery to the desired level. For example, in response to determining that the intensity is too low, the instructions may cause the smell delivery device to increase the flow rate.

Optionally, the processor is configured to use machine learning, neural networks, and/or artificial intelligence algorithms to process the sensor information to determine the validation of the delivery.

Optionally, the processor is configured to use machine learning, neural networks, and/or artificial intelligence algorithms to process the sensor information to determine the adjustment to the delivery. Analysis methods described above in relation to the eighth aspect can equally be applied here to determining the adjustment and to determining the validation. Simple threshold comparisons can be used to detect anomalies, or more complex algorithms such as pattern-matching may be used to confirm whether the smell is correct. For example, if the detected smell does not match the type of smell from the delivery information, an error can be outputted because the wrong canister may be inserted for example.

Optionally, the processor is configured to receive user information. The user information may be the same as the user information of the eighth aspect.

Optionally, the processor is configured to correlate the sensor information with the user information in determining the validation of the delivery. For example, along with correlating the sensor information and the delivery information, the user information may further be processed to determine the validation. In other words, the processor may be configured to correlate the sensor information with the delivery information and the user information. In other examples, the processor may be configured to correlate the sensor information or the delivery information with the user information. For example, the correlation may be used in identifying the smell. The user information can be used in conjunction with the sensor information to make a more accurate determination. The user information can also be used to affect the output to the user. For instance, if the user information identifies a relative level of smell ability of the user, the output may be affected. For example, if the user information identifies that the user can perceive a certain level of particular smells, the system may output the identification in a different manner (for example only outputting an image rather than a more invasive audio output), or may not output an identification if the determined intensity is above a threshold level above which it is known that the user can detect the olfactory output.

Optionally, the processor is configured to correlate the sensor information with the user information in determining the adjustment to the delivery. For example, based on historical user data, the processor may determine that the user has a low ability to smell this particular smell, so can adjust delivery to increase the intensity of the olfactory output to aid the user.

Optionally, the user information comprises an indication of one or more of the following, for a particular user: age; gender; ethnicity; other demographic information; health information; and/or historical sensor information. For example, historical sensor information may relate to a user's ability to detect one or more olfactory outputs. This can be used to determine whether the user will be able to, or will be likely to be able to, detect a particular olfactory output.

Optionally, the processor is configured to receive the user information from the smell delivery device, the smell sensing device, and/or an external database. For example, the smell delivery device or smell sensing device may contain a data storage containing user information, or may be able to access from an external database. The user information can then be forwarded to the processor. Alternatively, the processor may be able to obtain the user information directly by accessing a database locally or remotely.

Optionally, the smell sensing system is configured to collect user information relating to a user. In the same way as the eighth aspect, the information can be collated and used as historical data for that user, or combined with other users and possibly anonymized for general user data. The user information may be the same kind as the user information received by the processor. For example, the user information may identify sensor information for the user to be used as historical sensor information.

Optionally, the smell sensing system further comprises an environmental sensor configured to, in response to an environmental parameter, generate environmental information corresponding to the environmental parameter, and wherein the environmental sensor is configured to output the environmental information to the processor. The environmental information may be the same as the environmental information of the eighth aspect.

Optionally, the environmental parameter is one or more of: humidity; temperature; pressure; an identification or intensity of pollutants; a distance between the smell sensing device and the nose of the user; respiration of the user. Different environmental factors can affect a sense of smell, so by considering these, the accuracy can be improved. Humidity, temperature, pressure, and pollutants can each affect the background environment and the detected olfactory output in light of this. The position between the smell sensing device and the nose of the user can be used to determine how accurate the gas sensor detection is compared to that perceived by the user, where the closer the gas sensor the more accurate the measurement. In other words, the system may comprise a position sensor for detecting a distance between the smell sensing device and the nose of the user. The distance can also be used to compensate the sensor information, especially when the sensor is calibrated to determine a compensation for a given separation. The intensity perceived at the nose of the user can then be determined. The respiration of the user can also affect the detected olfactory output. The sensor may detect magnitude and direction of nasal flow, and timing of breathing. For example, magnitude and direction of nasal flow can affect how the olfactory output is measured. By compensating for this, the measurement can be more accurate. In other words, the system may comprise a respiration sensor for detecting respiration of the user. The environmental information may also be features of the gas sensor response to the olfactory output (chemical stimuli) which can be used to indicate the smell type. For example, the environmental sensor may be a temperature sensor and/or a humidity sensor.

Optionally, the environmental sensor comprises at least part of the smell sensing device. For example, the environmental sensor may be arranged on the smell sensing device. The environmental sensor may be integral or attached to the smell sensing device. This allows the compensation to be more accurate as the environmental sensor can be closer to the nose of the user and the gas sensor so is a better representation of the environment at the nose of the user. For example, the smell sensing device may comprise one or more gas sensors as well as one or more environmental sensors. For example, the smell sensing device may comprise one or more gas sensors and a temperature and/or humidity sensor. The one or more gas sensors may be an array of gas sensors, such as metal oxide sensors or a combination of metal oxide sensors and a PID sensor. Different metal oxide sensors can sense different smells, while the temperature and humidity sensors can compensate for the distortions in the gas sensor signal due to changes in humidity and temperature.

Optionally, the processor is configured to receive the environmental information.

Optionally, the processor is configured to correlate the sensor information with the environmental information in determining the validation of the delivery. For example, along with correlating the sensor information and the delivery information, the environmental information may be further processed to determine the validation. In other words, the processor may be configured to correlate the sensor information with the delivery information and the environmental information. In other examples, the processor may be configured to correlate the sensor information or the delivery information with the environmental information. This allows the environmental parameters to be taken into account when assessing the delivery of the olfactory output. For example, the smell sensing device may output sensor information indicating a low intensity of the olfactory output, which when correlated to the delivery information indicates an error in delivery, but in combination with the environmental information, the processor can determine that the drop in intensity is due to environmental parameters such as temperature or humidity. Together, this information can be used to more accurately validate delivery. In some examples, the processor may correlate the sensor information with the delivery information and the environmental information.

Optionally, the processor is configured to correlate the sensor information with the environmental information in determining the adjustment to the delivery. In the same manner as adjusting the delivery based on the sensor information and the delivery information, the delivery can be adjusted based on the correlation between the delivery information and the environmental information. For example, based on a particularly high temperature or humidity, the flow rate may be adjusted to compensate for the expected change in intensity. In some examples, the processor is configured to correlate the delivery information with the environmental information in determining the adjustment to the delivery.

In some examples, the processor may be configured to correlate the sensor information with the user information and the environmental information in determining the validation of the delivery. In some examples, the processor may be configured to correlate the sensor information with the user information and the environmental information in determining the adjustment to the delivery. For example, the processor may be configured to correlate the sensor information with the delivery information, the environmental information, and the user information. In other examples, the processor may be configured to correlate the sensor information, the delivery information, the environmental information, and/or the user information. By processing this together, the accuracy of the results can be improved. The validation and/or adjustment following correlation of these types of information may then be output to the user output device (for example to output the validation) and/or the smell delivery device (for example to adjust delivery).

According to a fifteenth aspect, there is disclosed a smell sensing system for validating a user perception of an olfactory output, comprising:
a processor;
a smell sensing device for detecting an olfactory output, comprising:
   at least one gas sensor configured to, in response to the olfactory output, generate sensor information corresponding to the olfactory output;
   wherein the smell sensing device is configured to output the sensor information to the processor; and
a user feedback device for responding to the olfactory output, comprising:
   a user input unit configured to, in response to receiving a user input indicating a user perception of the olfactory output, generate user input information corresponding to the olfactory output;
   wherein the user feedback device is configured to output the user input information to the processor; and
wherein the processor is configured to correlate the sensor information from the smell sensing device with the user input information from the user feedback device to determine a validation of the user perception of the olfactory output.

The smell sensing device and the user feedback device together can be used to correlate information to determine a validation of the user perception of an olfactory output. For example, the user feedback device can output user information corresponding to a user perception of an olfactory output (e.g. indicating whether the user can smell it or not) and the smell sensing device can output sensor information corresponding to the smell (e.g. confirming the presence of the smell). The processor can then validate the user perception of the olfactory output and determine whether the user perception is correct or not. For example, where the user cannot detect the olfactory output but the gas sensor detects a high intensity, the processor can determine that the user perception of the olfactory output is not correct. Further action can then be taken, such as informing the user. This can also be used to affect the output of an identification of the smell to the user. For example, if the user input information indicates that the user can correctly smell the olfactory output, an output of the identification may not be necessary. In another example, an incorrect user input may be used to identify a problem with the sense of smell or be used to feed into the user information stored that the user cannot sense that particular smell at that particular level or under those particular environmental conditions when combined with output from an environmental sensor, and the user may additionally be alerted via the user output device.

The fifteenth aspect may be provided in isolation. Features of other aspects may be applied to this aspect. The smell sensing device of the fifteenth aspect may be the same as the eighth or fourteenth aspects, and features of those aspects may be applied to the fifteenth aspect. The fifteenth aspect may also be provided in combination with other aspects. For example, the fifteenth aspect may be provided as part of the fourteenth aspect.

Thus, optionally, the smell sensing system of the fourteenth aspect may further comprise a user feedback device comprising a user input unit configured to, in response to receiving a user input indicating a user perception of the olfactory output, generate user input information corresponding to the olfactory output, wherein the user feedback device is configured to output the user input information to the processor.

The following optional features regarding the user input can therefore readily be applied to the fourteenth or fifteenth aspects.

Optionally, the user input comprises an indication of the user perception of one or more of the following: presence of the olfactory output; an intensity of the olfactory output; an identification of the smell or a type of smell associated with the olfactory output; a pulse duration of the olfactory output; a duration between subsequent pulses of the olfactory output; and/or whether the olfactory output is static or dynamic. Features of the user input may also be applied to the user input of other aspects (such as the eighth aspect) and vice versa. In one example, the user input unit is the user input unit of the user feedback device of the eighth aspect.

Optionally, the user input unit comprises one or more of: a button; a touch-screen; and/or a detector for detecting user response.

Optionally, the processor is configured to receive the user input information.

Optionally, the processor is configured to correlate the delivery information with the user input information in determining the validation of the delivery. This can be used in a similar manner to the sensor information, by verifying the output of the delivery. If the user input information indicates that the olfactory output is very low, but the delivery information indicates a high flow rate, an error may be triggered.

Optionally, the processor is configured to correlate the delivery information with the user input information in determining the adjustment to the delivery. An adjustment may also be made, for example based on the user input information indicating that they cannot detect the olfactory output, then the flow rate may be increased.

Optionally, the processor is configured to correlate the sensor information with the user input information to determine a validation of the user perception of the olfactory output. By comparing the user input to the sensor information, the processor can determine whether the user can correctly detect the olfactory output. For example, if the gas sensor detects a high intensity of olfactory output, but the user indicates that they cannot smell the olfactory output, then the processor can determine that the user perception is not correct. Remedial action may be taken such as output to the user. This may be used for smell testing or smell training where the user sense of smell can be verified.

In some examples, the delivery information may further be correlated with the sensor information and the user input information. For example, if the delivery information indicates a high flow rate, but the user input indicates a lack of or low intensity smell, then instead of assuming there is an issue with delivery, the sensor information may verify that the delivery was correct, and a high intensity was detected. This can then be used to infer that the user cannot detect the smell.

The user input information may further be correlated with other information. For example, the processor may correlate one or more of: the sensor information, the delivery information, the user information, the environmental information, and/or the user input information.

Optionally, the processor is configured to generate a user smell profile. The user smell profile may be the same as described above, such as in the eighth aspect.

Optionally, the user smell profile is indicative of one or more of: olfactory outputs perceived by the user; relative intensity of olfactory outputs perceived by the user; relative duration of olfactory outputs perceived by the user; effect of environmental parameters on olfactory outputs perceived by the user; base line of olfactory outputs from the user.

Optionally, the smell sensing system further comprises a user output device for outputting information to a user. The user output device may be the same as the user output device of other aspects such as the eighth aspect. For example, the user output device may contain a visual or audio output. The user output device may output information to the same user using the system, such as using the user input unit. In other examples, additionally or alternatively, the user output device may output information to a different user, such as a clinician performing a smell test on the user. In such cases, it may be beneficial that the user subject to the test does not directly receive the output in some cases.

Optionally, the user output device is configured to receive instructions from the processor, the instructions corresponding to the validation of the delivery of the smell by the smell delivery device. The instructions may cause the user output device to produce an output.

Optionally, the user output device is configured to, in response to receiving the instructions, output the validation of the delivery to the user. For example, this could confirm whether the delivery device is working or not.

Optionally, the user output device is configured to, in response to receiving the instructions, output the adjustment to the delivery to the user. For example, this could confirm what adjustments have been made, such as flow rate changes.

Optionally, the processor is configured to generate instructions corresponding to the validation of the delivery, and wherein the processor is configured to provide the instructions to the user output device.

Optionally, the processor is configured to generate instructions corresponding to the adjustment of the delivery, and wherein the processor is configured to provide the instructions to the user output device.

Features of other aspects may be applied to this aspect or vice versa. For example, features of the user output device of the fifteenth aspect may be applied to other aspects such as the eighth aspect.

The olfactory output could be provided by a smell delivery device, such as that described in the first aspect of this disclosure. Alternatively, other smell delivery devices or smell sources could be used. In some examples, the system includes a smell source instead of a delivery device. A smell source could be any object/device/system natural, organic or man-made that produces a smell. Jars or pens with liquids or solids that contain different smells could for example be used. Example of commonly recognisable smells could be lavender, lemon, rose, ginger, etc. Or mixtures of different smells or chemical compounds could also be used, where the mixture may be part of the olfactory white odour spectrum in which the dominant chemical compounds are no more recognisable. Alternatively smells could be identified by the user in relative forms using descriptors or characteristic commonly used even from other sensory domains (e.g. strong, sweet, pleasant, light, acid, loud) or through association with substances and possible object-related or object-source odours (e.g. food objects such as coffee, milk, cheese etc).

Disclosed herein, there is a smell sensing system, comprising:
a smell source for emitting an olfactory output; and
a smell sensing device for detecting the olfactory output delivered by the smell delivery device, comprising:
at least one gas sensor configured to, in response to the olfactory output, generate sensor information corresponding to the olfactory output;
wherein the smell sensing device is configured to output the sensor information to a processor.

The smell sensing system may then further comprise features of other aspects such as the fourteenth aspect. This may allow the sensing device and processing to be performed on a smell source that may be any object that emits an olfactory output, such as an odorant. This may be controlled such as by supplying a known concentration of olfactory output, or it may be natural and used in real-world scenarios where the smell source is not controlled (e.g. the smell of milk).

According to a sixteenth aspect, there is disclosed a method of operating the smell sensing system of the fourteenth aspect, the method comprising:
controlling the one or more airflow generating elements to transport the substance from the canister to the output component to deliver the olfactory output;
generating, by the at least one gas sensor, in response to the olfactory output, sensor information corresponding to the olfactory output;
outputting, by the smell sensing device, the sensor information to a processor.

Features of other aspects may be applied to this aspect. For example, features of the smell sensing system of the fourteenth aspect can be readily applied to the method of the sixteenth aspect. Ins some examples, the method further comprises performing operations associated with the smell sensing system of the fourteenth aspect. In particular, the method may comprise outputting user information, environmental information, and/or user input information to the processor. The method may comprise providing the processor and the processor performing operations. For example, the method may comprise the processor receiving the sensor information, processing the sensor information, and optionally generating and providing instructions to the delivery device, to validate or adjust the delivery. The method may comprise the processor receiving the user information, environmental information, and/or user input information. The method may comprise the processor processing or correlating the sensor information with the user information, the environmental information, and/or the user input information. The method may comprise the processor generating and providing instructions to the delivery device (for example validating or adjusting the delivery), based on the processing or correlating.

According to a seventeenth aspect, there is disclosed a processing device for validating a user perception of a smell, comprising:
a receiver configured to receive sensor information from a smell sensing device, the sensor information corresponding to an olfactory output detected by the smell sensing device;
wherein the receiver is configured to receive user input information from a user feedback device comprising a user input unit, the user input information corresponding to a user input indicating a user perception of the olfactory output; and
a processor configured to correlate the sensor information from the smell sensing device with the user input information from the user feedback device to determine a validation of the user perception of the smell.

It will be appreciated that the processing device of the seventeenth aspect is complementary to the system of the fourteenth aspect. Features of other aspects may be applied to this aspect. The processing device may be further configured in a similar manner to the processor disclosed herein, and features of the processor of the fourteenth aspect may be readily applied to the processing device of the seventeenth aspect. For example, the processor may be configured to correlate information as described above or use machine learning or other algorithms disclosed herein. The receiver and transmitter may be analogous to those disclosed herein, and may be the same component configured for receiving and transmitting or may be separate components. For example, the receiver and transmitter may be wireless for wirelessly communicating with the smell sensing device and the user output device.

According to an eighteenth aspect, there is disclosed a method of validating a user perception of a smell, comprising:
receiving sensor information from a smell sensing device, the sensor information corresponding to an olfactory output detected by the smell sensing device;
receiving user input information from a user feedback device comprising a user input unit, the user input information corresponding to a user input indicating a user perception of the olfactory output; and
correlating the sensor information from the smell sensing device with the user input information from the user feedback device to determine a validation of the user perception of the smell.

Optionally, the method is computer implemented. The method may be performed by a processor such as the processor disclosed in the eighth or fourteenth aspect or the processing device of the seventeenth aspect. Features of other aspects may be applied to this aspect. Features of the eighth aspect or fourteenth aspect may be readily applied to the method of the eighteenth aspect. Method features of the processor can readily be applied to the eleventh aspect, such as receiving and processing other information such as user information, environmental information, user input information, and sending instructions based on such processing.

According to a nineteenth aspect, there is disclosed a computer program, computer program product, or computer-readable medium comprising instructions which, when executed by a processor, cause the processor to perform the method of the eighteenth aspect. Thus, the computer program may be in the form of software such as an application (or app). Features of other aspects may be applied to this aspect. Features of the eighth or fourteenth aspect may be readily applied to the nineteenth aspect.

According to a twentieth aspect, there is disclosed a computing device comprising the computer program, computer program product, or computer-readable medium of the nineteenth aspect. For example, this may be a computer (e.g. desktop, laptop), a smartphone, a tablet, or other smart device (e.g. smart watch etc.) storing the computer program such as in the form of a software application. In another example, a computer-readable medium such as a computer data storage (e.g. hard drive) may be provided within a computing device. Features of other aspects may be applied to this aspect. Features of the eighth or fourteenth aspect may be readily applied to the twentieth aspect.

Features of any aspect may readily be combinable with features of another aspect. Product features may readily be applied to method features and vice versa.

The advantages described in the present invention are presented in the detailed description with the accompanying figures.

### BRIEF DESCRIPTION OF THE FIGURES

Some details of the present invention, both as to its components and operation, are given in the accompanying drawings.

FIG. 1 shows a schematic diagram of a first embodiment of a smell delivery device. The smell delivery device shown in FIG. 1 comprises a single air flow channel.

FIG. 2 shows a schematic diagram of a second embodiment of a smell delivery device. The smell delivery device in FIG. 2 comprises a plurality of air flow channels.

FIG. 3 shows an external view of an embodiment of a smell delivery device. The smell delivery device of FIG. 3 is shown from a user, or operator perspective, showing only those features visible from the exterior of the smell delivery device.

FIG. 4 illustrates schematically a cross-sectional view of the smell delivery device of a third embodiment.

FIG. 5 illustrates a realistic appearance of the internal workings of the air flow system in an exploded view of the smell delivery device from the air input to the output according to a further embodiment.

FIG. 6 shows a realistic external appearance of the smell delivery device in accordance with the embodiment shown in FIG. 5.

FIG. 7 illustrates a further schematic of external appearance of the smell delivery device according to a further embodiment. FIG. 7 is based on FIG. 3, but optional external sensors and communication means are further shown. External components are shown, including an input of the power supply, LCD location and example of possible connectivity platforms and a possible location for a distance sensor.

FIG. 8 illustrates schematically a side-view of the air flow path from an air flow generator(s) to the outputs.

FIG. 9 shows a block diagram of the adaptive smell delivery system unit. The smell delivery device, user, user feedback, and adaptive system unit are shown. The communication between these units is also shown.

FIG. 10 shows a block diagram of adaptive smell delivery system unit embedded in a possible cloud-based configuration.

FIG. 11 shows a system configuration where the delivery device is in a different physical space and connected through the Internet (using a client/server architecture) to a remote computer, as in the cloud or in-premises. This example configuration could be applicable to a numerous Internet-of-Things applications.

FIG. 12 shows a further system configuration where the delivery device connects directly (via USB port for serial communication, Bluetooth, Wi-Fi, RF technologies, etc.) to a device used as orchestrator with other devices in the same physical location (like audio-visual screen, VR sets, haptic devices, light systems, etc.) to achieve a low latency integration and deliver a tight coordinated multisensory experience.

FIG. 13 shows a further system configuration where the delivery device, as shown in FIG. 10, is used differently in such a way that the orchestrator connects to a remote server through the Internet (to deliver information, obtain instructions, etc.).

FIG. 14 presents a possible system configuration where the delivery device is connected through the Internet to a remote computer that may be provided by the cloud or by in-premise servers to deliver a general experience that does not require input from the user.

FIG. 15 shows a block diagram of a further system configuration where the delivery device functions in isolation based on an application running on its own automated delivery device control unit.

FIG. 16 shows a method of delivering a smell from the smell delivery device.

FIG. 17 shows a flow diagram illustrating a first example of a process of a smell delivery method.

FIG. 18 shows a flow diagram illustrating a second example of a process of a smell delivery method.

FIG. 19 shows a flow diagram illustrating a third example of a process of a smell delivery method.

FIG. 20 shows a block diagram of an example of an adaptive smell delivery system together with a smell sensing device. The smell delivery device, user, user feedback, and adaptive system unit are shown. The independent unit of a smell sensing device placed in the proximity of the user is also shown. The communication between these units is also shown.

FIG. 21 shows a block diagram of an example of an adaptive smell delivery system and an independent unit of a smell sensing device embedded in a cloud-based configuration.

FIG. 22A shows a schematic representation of examples of the various components involved in smell testing or smell training.

FIG. 22B shows an example smell sensing device attached to glasses.

FIG. 22C shows an example clip-on smell sensing device.

FIG. 23 shows a 3D schematic representation of an example smell sensing device comprising a printed PCB, multi-sensing VOC sensors, battery, ASIC, and interface ports.

FIG. 24A shows the smell transient signal from a PID sensor incorporated in an example smell sensing device.

FIG. 24B shows the decay of the concentration of the smell as a function of the distance between the smell delivery device and the smell sensing device.

### DETAILED DESCRIPTION

There is described a smell delivery device comprising a delivery channel for receiving a substance from a canister, the substance configured to produce an olfactory output, such as a smell, an output component through which the substance is emitted, and one or more airflow generating elements configured to generate airflow to transport the substance from the canister to the output component. A flow controller is configured to control the flow rate, or concentration, of the substance through the delivery channel to the output component. The flow controller is configured to control the flow rate, or concentration of the substance from the delivery channel to the output component in response to feedback from at least one of a sensor configured to sense the flow rate, or concentration of the substance, through the delivery channel, and/or, an environmental sensor configured to sense environmental conditions, and/or a user feedback device configured to receive an input from a user.

There is herein disclosed a smell delivery device that may be used in medical applications, to test the smell performance of patients or train the smell perception of users. Such smell delivery device according to the main aspect may also be used in applications such as entertainment, or consumer products/services to deliver and control the smell in the ambient surrounding the device.

This smell delivery device is a hardware solution for accurate and precise administration of smell stimuli or mixtures of such smell stimuli, with low latency and no-cross contamination between stimuli.

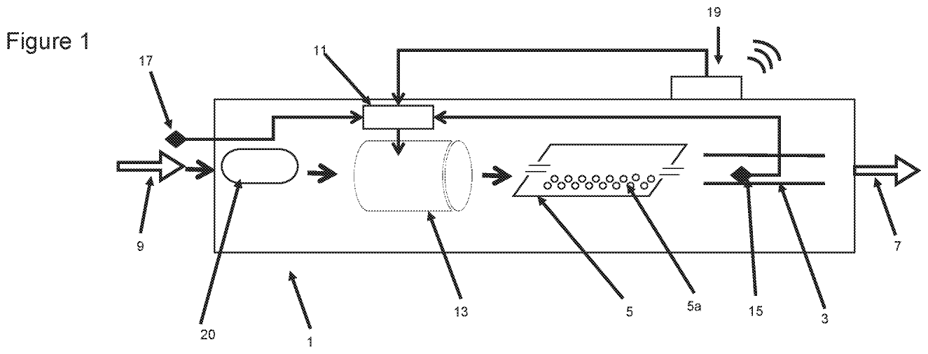

FIG. 1 shows an embodiment of the invention. In particular, FIG. 1 shows a working embodiment in which just a single canister 5 is present, and therefore there is a single air flow pathway. The smell delivery device comprises a canister 5 containing a substance 5a, a delivery channel 3, a sensor 15 to measure the flow rate/concentration of the substance in the delivery channel 3, an output component 7, a flow generator 13, a flow controller 11, an air inlet 9, an air filter 20, an environmental sensor 17, and a user feedback device 19.

Air is drawn into the air inlet 9 by the flow generator 13. In this embodiment the air flow then travels through the air filter 20, the canister 5, the delivery channel 3 and then out through the output component 7. The sensor 15 configured to measure the flow rate/concentration of the substance in the delivery channel 3 is positioned within the delivery channel 3. The environmental sensor 17 is positioned on the exterior of the smell delivery device. The user feedback device 19 is positioned anywhere on the smell delivery device. The sensor 15 configured to measure the flow rate/concentration in the delivery channel, the environmental sensor 17, and the user feedback device 19 are all configured to communicate with the flow controller 11. The air flow controller 11 controls the flow rate created by the air flow generator 13. The user feedback device 19 is configured to receive an input from a user.

The air inlet 9 forms an entrance for air to enter the smell delivery device 1. Air is drawn into the air inlet by the flow generator 13. This brings airflow into the device 1. The air flow, after being brought through the air inlet 9, is directed to an air filter 20. This removes impurities from the air, in order to minimise contamination.

The substance 5a is then added to the purified air flow. In this embodiment the canister 5 comprises polymer beads saturated with the substance 5a. In this embodiment air flow is directed through the canister 5 such that the air makes contact with polymer beads, such that some of the substance 5a is drawn into the air flow. The amount of the substance that is drawn into the air flow is predictable based on the strength of the air flow.

The air flow comprising the substance 5a then travels through the delivery channel 3, and out of the output component 7. The user is positioned at/near the output component, and then may experience an olfactory output associated with the substance 5a, such as a smell.

As the air flow travels through the delivery channel 3 the sensor 15 measures the flow rate/concentration of the substance in the delivery channel 3. This is then compared with a target flow rate/concentration by the flow controller 11. If the measured flow rate/concentration is lower than the target the flow controller 11 will instruct the flow generator 13 to increase the air flow. If the measured flow rate/concentration is above the target flow rate the flow controller 11 will instruct the flow generator 13 to decrease the air flow. In this manner the air flow is modulated and a precise flow rate of the substance is maintained.

Additionally, the smell delivery device 1 comprises the environmental sensor 17. The environmental sensor 17 in this embodiment is configured to measure the humidity and temperature of the environment. During usage if one of these parameters changes the flow controller 11 instructs the flow generator 13 to modulate the flow rate. For example, in some embodiments if the humidity increases then the flow rate may be modulated by increasing the flow rate, and so the flow rate/concentration of the substance.

Additionally, the smell delivery device 1 comprises the user feedback device 19. As the device is in operation the user provides feedback. For example, in this embodiment the device 1 is used to determine the concentration of a substance at which a user can perceive its smell. The user feedback is therefore an indication of whether the smell is perceived. Starting from a low concentration/flow rate the user provides feedback indicating that they cannot perceive the smell. The flow controller 11 then instructs the flow generator 13 to modulate the flow rate/concentration of the substance to increase the flow rate/concentration. The flow rate/concentration then iteratively reaches the tipping point at which the smell is perceived by the user. In other embodiments this may be reversed with the flow rate/concentration starting at a high level and being reduced until the user can no longer smell the substance.

It is noted that the canister 5 may be a replaceable element, and therefore the smell delivery device 1 may be manufactured without a canister 5 present. The canister 5 itself may comprise a storage volume storing a substance, wherein the substance is associated with an olfactory output. The canister 5 is configured to be received by the smell delivery device 1, and wherein once received within the smell delivery device 1, the canister 5 is configured to emit the substance into the delivery channel 5. For example, the canisters 5 may be spring-loaded into position such that they can be removed and replaced with a single click. Alternatively, the canisters 5 may slot into place.

It is noted that FIG. 1 merely shows one example of a working embodiment. Certain features described above are not essential. For example, in some applications the air filter 20 is not needed as air drawn in through the air inlet is of sufficient purity. Further, the air inlet 9 itself may be inessential. Some embodiments may instead comprise a compressed air store. The flow controller 11 in such embodiments may control the flow of compressed air from the compressed air store. It is also noted that it is only essential that one sensor of: the user feedback device 19, the environmental sensor 17, or the sensor 15 configured to measure the flow rate/concentration of the substance in the delivery channel, is present. It is also noted that the canister 5 may be a replaceable element. Therefore, when manufacturing and selling a smell delivery device the canister 5 may not be present in the smell delivery device.

The flow generator 13 may optionally be a pump, or a valve. For example, the flow generator 9 may comprise a proportional valve such as a piezoelectric or solenoid based proportional valve. Alternatively, the flow generator may comprise a pump, wherein the power of the pump may be controlled. For example, the pump may comprise a piezoelectric, piston or diaphragm type pump in which the power of the pump is controllable.

The environmental sensor 17 may comprise any sensor that measures a property of the local environment. For example, the environmental sensor may comprise a thermometer to measure a temperature, a barometer to measure pressure, a hygrometer to measure humidity, or a gas sensor to identify background contamination in the environment. In the case of the gas sensor, the system may then shutdown if the contamination level was too high, for example above a pre-set threshold.

The sensors 15, 17, 19 may sample at a constant rate. Alternatively, if the sensors detect a measurement close to a threshold they may increase the sampling rate to ensure any breach of a pre-set threshold is detected quickly.

The sensor 15 may be configured to measure the flow rate/concentration of the substance in the delivery channel may either comprise a flow rate sensor or a concentration sensor. For example, the sensor 15 may comprise a gas sensor. Gas sensor types could include metal oxide, photo-ionisation, mass spectrometry, ion-mobility spectrometry and electro-chemical. An e-nose could be used to identify different scent types. Alternatively, a pressure sensor, or a stress/strain gauge may be used to monitor the flow rate through the delivery channel. Monitoring the flow rate of the substance through the delivery channel 3 may comprise monitoring the total flow rate through the delivery channel 3, as this is indicative of the flow rate of the substance through the delivery channel. Alternatively, or additionally, the mass of the substance contained within the canister 5 may be monitored and this may be used to determine the flow rate of the substance through the delivery channel 3.

The canister 5 may be any canister 5 containing a substance 5a that provides an olfactory output. The example shown illustrates polymer beads saturated with a substance 5a, however other configurations may be used. For example, the canister 5 may contain a liquid, powder, or gel comprising the substance 5a. In the case of a powder the substance 5a may be carried by the air flow. In alternative embodiments the substance 5a may be vaporised by a valve and introduced into the air flow.

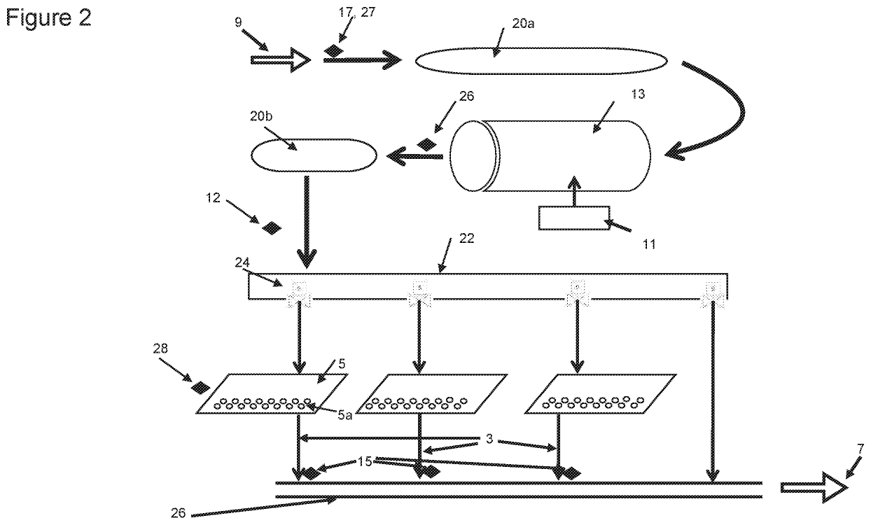

FIG. 2 shows an embodiment in which further canisters are present. The communication between the sensors and the flow controller 11 are not shown in FIG. 2 for simplicity. The smell delivery device of FIG. 2 comprises a plurality of canisters 5, each containing a substance 5a, a delivery channel 3 associated with each canister 5, a sensor 15 to measure the flow rate/concentration of the substance in each delivery channel, a manifold element 22, and a plurality of valves 24, wherein each valve is associated with a respective canister 5, an output component 7, a flow generator 13, a flow controller 11, an air inlet 9, an air filter 20a, 20b, an environmental sensor 17, and a user feedback device 19. A further valve can control the supply of clean air to dilute the concentration of the substance (this is an optional feature).

Air is drawn into the air inlet 9 by the flow generator 13. In this embodiment the air flow then travels through the air filter 20a. The air then passes through the flow generator 13 and is then directed towards a manifold 22. A manifold 22 is a pipe or chamber with several openings. In this case each opening is controlled by the flow controller 11 and may constitute a valve 24. The valves 24 are opened dependent upon where the flow controller 11 determines the air flow should be directed. These valves may be simple on-off types, or proportional valves to individually control the flow rate in each channel. Each valve 24 is associated with a respective canister 5, such that, when open, air passing through a first valve 24 will travel through a first canister 5, and air travelling through a second valve 24 will travel through a second canister 5, etc. Each canister 5 has associated with it its own delivery channel 3. Therefore, air travelling through a canister 5 will travel through its associated delivery channel 3. This ensures that the substances 5a stored in each canister are isolated from one another. The delivery channels 3 may, as shown in FIG. 2, meet shortly before the output component 7. This may enable substances, and therefore their associated smells, to be mixed together, in order to form new smells. It may also disguise a change in the canister being used, so that the user cannot determine that the smell has changed without actually perceiving the change (the user may otherwise notice that air if being output from a new output, and associate that with a changing smell). The delivery channels may be separate, and each have separate output components 7. These components may then be joined by an output extension 26. For example, an output extension may terminate in a mask to be work by the patient so that the mask is replaceable after each use to increase hygiene, which also avoiding bias in perception by the user. The substances emitted may then be combined in the output extension 26 in such embodiments. The environmental sensor 17 is positioned on the exterior of the smell delivery device 1. The user feedback device 19 is positioned anywhere on/in the smell delivery device (and is not shown in FIG. 2 for this reason). A sensor 15 configured to measure the flow rate/concentration of a substance through a delivery channel is located in each delivery channel 3. The sensor 15 configured to measure the flow rate/concentration in the delivery channel, the environmental sensor 17, and the user feedback device 19 are all configured to communicate with the flow controller. The air flow controller 11 controls the flow rate created by the air flow generator 13. Alternatively, the air flow generated may be constant, and it may be the position of the valve 24 in the manifold 22 that is controlled by the flow controller 11, and enables the flow rate through the delivery channels 3 to be controlled. The user feedback device 19 is configured to receive an input from a user.

It is noted that a single flow generator 13 may be used to produce flow from a plurality of delivery channels. However, each delivery channel 3 may also have associated with it a flow generator 13. Similarly, a single flow controller 11 may be used, or alternatively each delivery channel 3 may have associated with it an individual flow controller 11.

Each canister 5 may contain a substance 5a. In some embodiments these substances 5a may differ from one another. In another embodiment one or more of the canisters 5 may be empty such that only air is emitted by the associated delivery channel 3 (this is shown by the lack of a canister associated with the fourth valve in FIG. 2). This may allow the concentration of the substance 5a emitted by the smell delivery device 1 to be further modulated. For example, in one embodiment a first delivery channel 3 may be emitting a first substance 5a. It may be advantageous to create a square shaped concentration graph over time of the output of this substance 5a. By switching the output of a second delivery channel 3, emitting only air, off and on this reduces the concentration of the substance 5a when the second delivery channel is on, and does not affect the concentration when the second delivery channel is off. Therefore, a square shaped emission curve may be generated.

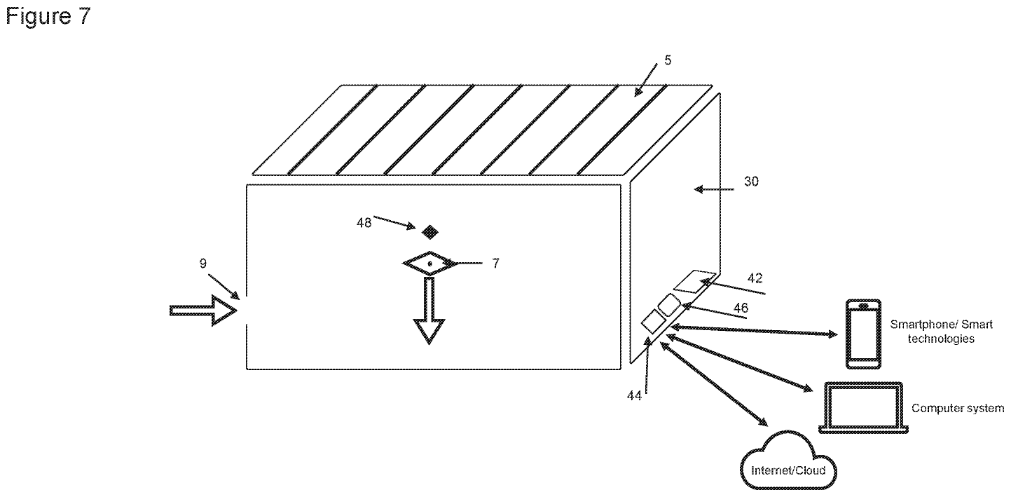

FIG. 2 also illustrates possible positions where the main system sensors may be located, as for example along the air flow path or in the canister array or external to the delivery enclosure. The device may sense information though at least a pressure sensor 26 that reads the flow generator 13 (or pump) pressure and controls the flow through one or multiple delivery channels 3. There may also be a temperature and humidity sensor 17, air flow sensors 15, and a gas detector sensor 27. The gas sensor could be a non-selective type (e.g. photoionization or metal oxide sensor) which would respond to a range of odours. Alternatively, a selective gas sensor could be used (e.g. an e-nose or ion-mass spectrometer) to discriminate between odour types. The specifications and types of the sensors are defined by the application requirements however, pressure and temperature sensors may be particularly advantageous as part of the feedback loop system. The feedback loop system may be part of the delivery device control unit for modulating and controlling the hardware components illustrated in FIG. 2 and FIG. 7 for the delivery device control unit two main systems.

The sensors for characterization of the chemical substances used in the cartridges may be recording data to compute the smell delivery parameters for an optimal application performance. The type of sensor for such a chemical characterization may be of different types determined by the application requirements, such sensor could be gas sensors or embedded PID (photoionization detector) or odour sensors.

An array of various sensors or free-located inside and outside the delivery device 1 various types may be included inside the smell delivery device, in different locations. The specific type and number of sensors to integrate might be determined through considering key factors such as sensitivity range, stability, response time, sample frequencies, and durability in relation to the specific application requirements.

Monitoring the consumption performance and the status of the chemical substances in the cartridges may be advantageous. Such monitoring may be performed by at least one weight sensor 28 located in each of the cartridges 5, as illustrated in FIG. 2. The weight sensors 28 could be of different types, and may for example comprise a strain gauge or a capacitance sensor or a hydraulic sensor, or a pneumatic sensor, which could detect changes within a physical force, pressure or weight, and produces an output that is comparative to the physical stimulus. The types and specifications of the weight sensors 28 could be determined by the nature the chemical substances, liquid, gel or powder, and the application requirements. Alternatively, the dielectric properties of the chemical substances present in the cartridges could be sensed, through electrodes located in the cartridges' walls, to indicate changes in the usage of the chemical compounds over time.

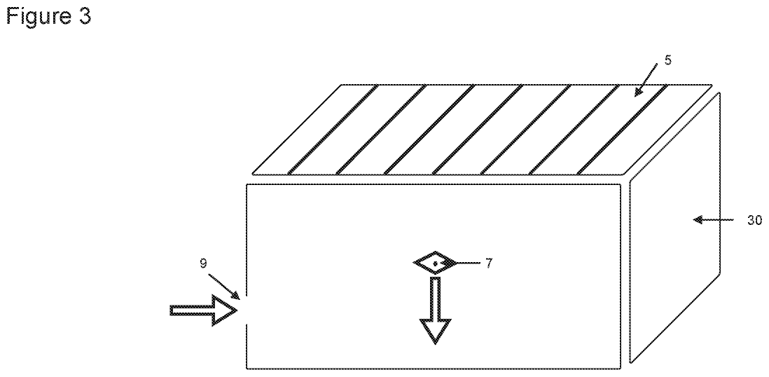

FIG. 3 shows a schematic external view of an embodiment of a smell delivery device. This is the view that a user, or operator may have of the device. FIG. 3 shows an air input 9 into the smell delivery device 1 and the output component 7 for emitting an air flow transporting the chemical substances (i.e., smell stimuli). The output air flow may contain substances from one or more canisters 5, and these substance may be in different chemical states (i.e., liquid, powder, gel etc.). The smell stimuli are transported from the containers (e.g., bottle or cartridges) housing the chemical substance to the air output location 7. The walls of the enclosure 30 are also shown.

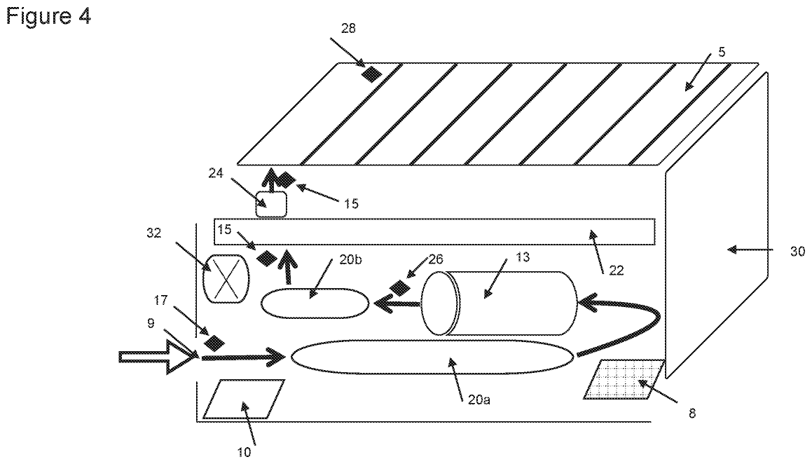

FIG. 4 illustrates schematically a cross-sectional view of the smell delivery device according to a possible embodiment. FIG. 4 shows the possible locations and specifications of the different sensors 15, 17, 19, 28 which is determined by the different requirements applications: an enclosure 30, which may be produced of possible different materials (e.g., plastic, aluminium), with at least one hole comprising an air inlet 9 to allow external air to be input into the system 1. The delivery device 1 presented in the present disclosure may have different shapes and dimensions. FIG. 4 also shows air input 1, the enclosure surrounding the device 30, canisters 5, a first filter 20a, and a second filter 20b, a first electronic control unit 10, and a second electronic control unit 8 (these may instead be replaced by a single flow controller 11), a flow generator such as a pump 13, a fan 32, a valve 24, a pressure sensor 15, an environmental sensor 17 configured to measure temperature and humidity, a weight sensor 28 to measure the weight of the canisters, and a manifold 22. The enclosure 30 may be used to surround the internal working of the device, and to act as a casing.

The canisters 5 are shown schematically in FIG. 4. The first filter 20a and second filter 20b may comprise carbon filters, and are positioned before and after the pump 13, in order to purify and remove any detritus from the incoming air. The pump is a form of a flow generator 13, and other flow generators may alternately be used. The fan 32 is configured to prevent the other components of the device, such as the flow generator 13, or the valves 24 from overheating. The pressure sensors 15 detect pressure on entry to the manifold 22 and in the delivery channels, whilst valve 24 determines whether air in the manifold is transferred to cartridge 5. The outlet is not shown in FIG. 4. The air flow path of FIG. 4 is substantively the same as that shown in FIG. 2.

A smell delivery device 1 may be defined as an enclosure 30 with air intake 9 from the environment to allow flow generation. The flow generated is the fundamental carrier of the chemical substances. This flow should be directed at least to one valve 24 connected with one cartridge 5 with at least one air output component for smell stimuli 7. The air output component 7 allows the carrier to deliver the smell (chemical substances/odorants) to a person's nose, or within a person's head space. The smell output 7 may have a number of different shapes and dimensions, which would define the speed and spatial resolution, according to the users' specified or adapted distance. For example, a circular smell output component 7 with a diameter of 1 mm would generate a high-speed delivery with an accurate spatial resolution and dispersion emulating a conical trajectory. The smell output connotations may be functional in relation to the optimal perceptual point and the user' distance and position, so that the users' nose or head space may be reached by the carrier with the smell stimuli. These distances may be calculated in function of contextual and environmental factors. As such the timing of the smell delivery, the air flow, and the pressure of the air flow, could be calculated in relation and adapted to suit the environment and chemical substances used. The delivery range may vary between 10-45 cm, which is defined by the selected delivery device parameters and the type of flow generator 13.

The air output 7 for smell stimuli could be a supra-perceptual threshold stimulus (i.e., the smell stimulus is above the weakest stimulus perceivable) or sub-perceptual threshold stimulus (i.e., the smell stimulus is below the weakest stimulus perceivable). The perceptual threshold stimuli may be defined by application requirements and computed within by a processor. The processor may be located in a separate device, such as an adaptive system unit in communication with the smell delivery system.

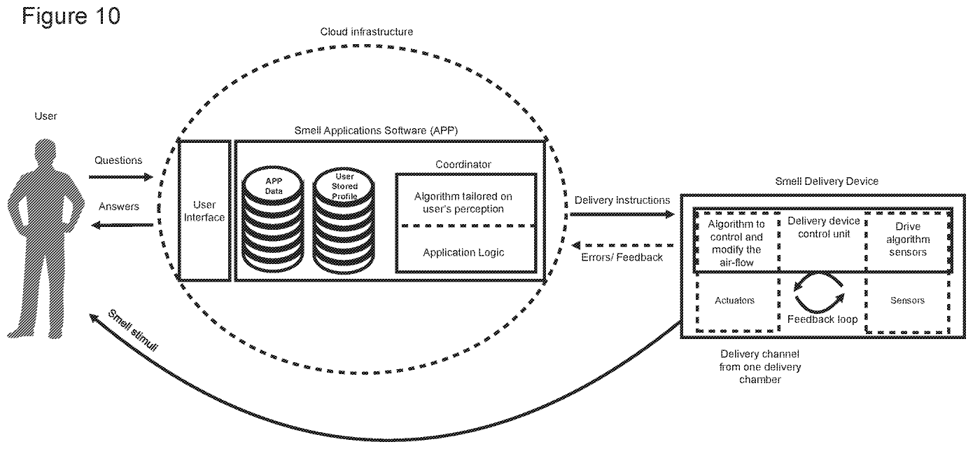

Two custom control units (PCB-printed circuit boards) are shown, one electronic unit control 10 may drive the pump's behaviour and activate at least one fan 32, with inputs from a least a differential/gauge pressure or flow sensors which is attached directly or through a bypass to the at least one channel, and a temperature sensor and humidity sensor. A second electronic unit control 8 may receive and analyse the outputs from the sensors may drive the valve activity, with inputs from the user feedback device (e.g. answers or preferences) chemical substances information for the data stored in the smell application software (see FIG. 7), distance sensor 34 and weight sensors 28. The possibility of data storing may be local on an internally accessible storage card and/or remote in a cloud-database storage, in which single user profiles may be generated and adapted for different Smell Applications Software (APP), as presented in FIG. 10.

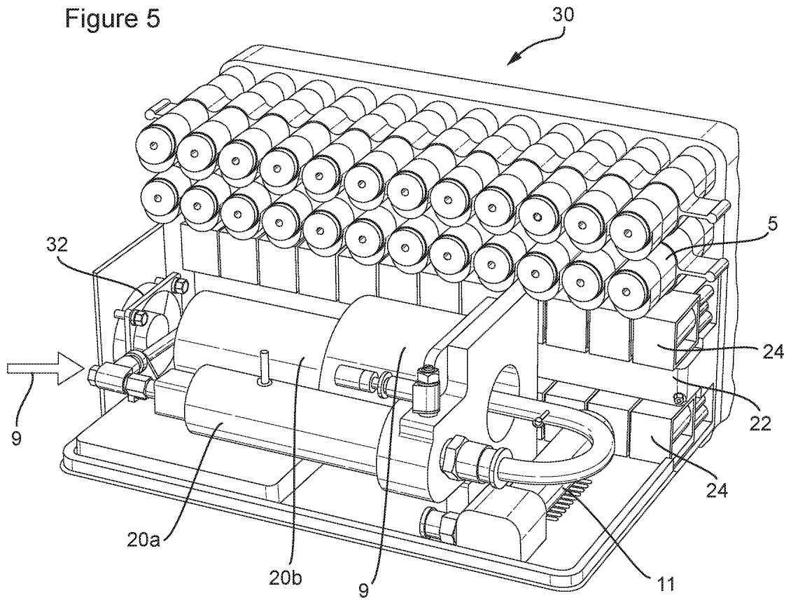

FIG. 5 shows a further embodiment of the smell delivery device (in cross-section) comprising: two parallel arrays of each 12 cartridges 5 with a 3 mm connector to a manifold 22 and the 24 solenoid valves respectively 24, within an aluminium and plastic enclosure 30 of specific dimensions 25 cm length, 18 cm width, 15 cm height and 7 kg weight, air input, a flow controller 11, two carbon filters (20a, 20b), a pump or flow generator 13, a fan 32 and an air input 9. FIG. 5 shows a realistic view of how one embodiment of the present invention may appear. The small size, and portable nature of the device 1 is particularly advantageous. This is achieved by using the arrangement shown in FIG. 5. In particular, the air flow is configured to bend round through the device 1, such that the size of the smell delivery device may be reduced, without reducing the length of the air flow path. The canisters 5 are grouped together in a regular array to save space, and to efficiently connect the manifold 22 with the canisters, via valves 24. A single manifold is shown with two banks of valves 24 attached to further save space, and maximise the use of the internal area of the manifold 22.

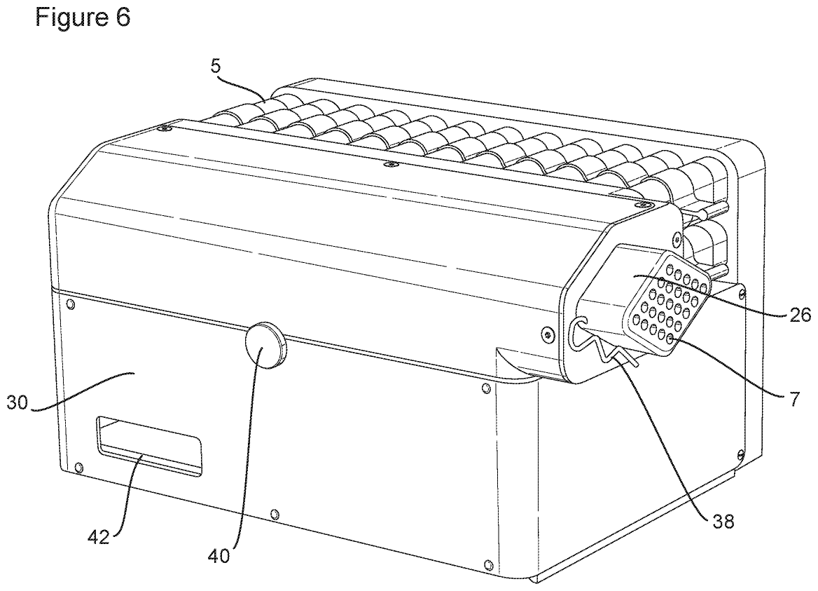

FIG. 6 shows a realistic external view of the smell delivery device presented in FIG. 5. In this example the enclosure 30 is formed from plastic material with structural aluminium inserts comprising: a possible frame for a display 42 (such as an LED display), an output extension 26 removable and exchangeable through a magnetic knob 40 and an output component 7, both with possibility to be exchanged by the means of an ad hoc key 38. The magnetic knob 40 may be configured to anchor the output extension in place, whilst also allowing for easy removal of the output extension when required. The smell output component 7 is shown with 24 different delivery channels 3, one for each canister 5 shown. The output extension 26 may be attached and removed as required by the user. The output extension may enable smells emitted by the 24 delivery channels 3 shown at the output component to be mixed together before being perceived by the user. In this manner new smells may be created or diluted through combining those associated with the substances in each canister. The ad hoc key may enable the output extension to be locked in place. The ad hoc key may be a screw like member, or alternatively may simply comprise a mount.

FIG. 7 shows a schematic diagram of an external view of a smell delivery device 1. The enclosure 30, air input 9, and canisters 5, and output component 7 are shown (as also seen in FIG. 3). However, additionally, elements 42, 44 and 46 are shown. These are optional features comprising a display 42 such as an LED or LCD display, an external power supply unit 46, and a connection element 44. The external power supply unit 46, may for example be a replaceable battery unit, or may comprise a plug socket. The display 42 may show the amount of charge remaining, or the time the device can be used for before a next charge. Any errors may also be shown on a display unit 42. Alternatively results of any smell test may be shown on the display. The connection element 44 may be used to enable the smell delivery device to be connected with other devices. The connection element 44 may be a physical connection, such as a USB port. Alternatively the smell delivery device may be connected wirelessly using known standards such as Bluetooth, or Wi-Fi, or other RF technology.

A distance sensor may be used to infer the location of the user 48, therefore inputting the information to the control unit for the adjustment of the delivery parameters accordingly. The type of distance sensors 4 would be defined in relation to the target application(s). These sensors may be an optical distance sensor (LIDAR), a CO2 sensor or ultrasonic sensor. The CO2 type sensor, for instance, may allow the number of people in the vicinity of the device to be estimated through detection of the CO2 level.

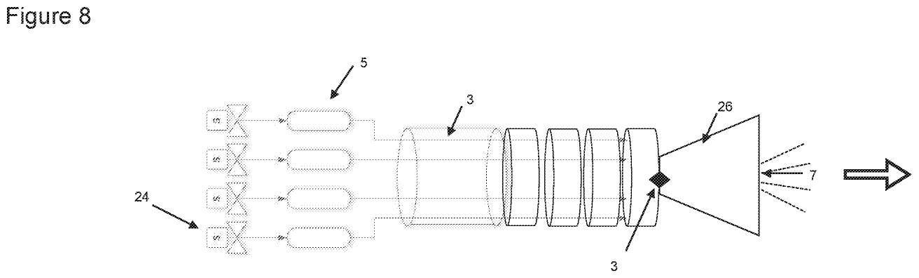

FIG. 8 schematically illustrates the air flow path from the valves 24 into the sealed cartridges 5 through single isolated channels 3 leading to the smell output component 7. Alternately, the smell output may have an attachable output extension 26 of a various length, for example 45 cm via PTFE-F piping wrapped in a cover of different material (e.g. plastic, aluminium, cord). For example, with a single channel of a 4 mm diameter, 2.4 mm bore size, the extension may be connected with a 3D printed-support or another type of material and design. The extension 26 may have a function to contain and wrap the single channel, from one to many, or may be an individual extension of single PTFE-F piping. The single piping extension could have the possibility to have diversified locations for the delivery device smell output. For example, with the single piping setup, the delivery range could cover long distances, more than 2 m. The single piping extension may be connected to a 3D printed-support for user's head space or nose-directed diffusion.

The extension length and type may be defined by the applications requirements and scenarios.

The air input intake 9 may be immediately passing through a first of two coalescing carbon air-filters 20a, 20b to remove external environmental particles and odours. The device may have a generic flow generator 13 such as a pump to produce and control the air flow sourcing the filtered air. The flow generator may be of different types, an axial piston, membrane pump or micro-pump or piezoelectric pump, the different applications and implementation requirements would define the type of flow generator most suitable.

The air flow generated by the pump system might be conveyed through a sealed pipe system to at least one two-way valves 24 such as solenoid valves or piezoelectric type valves, after being carbon filtered to remove additional particles and odours from the flow generator 13 by the means of a second coalescing filter 20b. The air flowing generated after the second filter would produce a breathable air flow that may be odourless with absence of particles.

Multiple solenoid valves might be served from the same pump system though a manifold structure 22 with a modular access points for several one-way valves 24. Each valve-type solenoid may be fully closed or accurately opened and regulated to modulate the direction and speed of the flow towards each single cartridge 5. The device may have at least one or more cartridges 5, each of them with a weight sensor 28 to monitor changes in mass. FIGS. 1 and 2 illustrates schematically a schematic of the air flow path from the intake input to the smell output, presenting the main components involved in this process.

The delivery device components and the flow generator may overheat, hence at least one axial fan (32, FIG. 4) can be used to reduce the internal temperature. The fan behaviour may be defined by the temperature sensor and be controlled by the delivery device control unit (see FIG. 7). The behaviour of the fan may be simplistically activated in connection of an internal temperature range (above 40° C.) and external range (above 38□C) or in function of the time of activation or the number of consecutive deliveries of smell stimuli.

The air flow may be measured with an external flow meter (1 SLM-10 SLM, where SLM means Standard Litre per Minute) that could be a digital type as a clamp-on ultrasonic flow meter or air speed sensor or embedded in smell delivery device with a flow sensor 15 or air speed sensor. The flow sensor may have a resolution of 50 SCCM (where SCCM means standard cubic centimetre per minute). The air flow may be modulated through the pump or solenoid behaviours, with accuracy or automated feedback loop control. The air flow for each channel 3 may range between ˜0.2 L/min to ˜6 L/min, with an air flow target range from the flow generator of ˜0-8 SLM, the range may be determinate specifically for the application scenarios driving the definition of the flow generator types.

The overall delivery device might have a low audible mechanical noise for bias avoidance of the user, such as when used in perceptual studies or health applications. The audible noise may vary in function of the flow type generators, defined and adapted to application requirements. In the case of the healthcare applications, as smell test or training, the noise would be below 40 dB using a diaphragm pump type while for entertainment the noise level requirement can be less prominent and therefore a piston pump could be applicable. In entertainment applications, the delivery device may be positioned away from the users or in a acoustically treated box.

The device may have at least 1 delivery output connected with one canister (at least with a volume of 5 mL) that could be made from various non-porous and odourless material, containing a chemical substance in different forms. The chemical substance may be in a different form in function of the application scopes and requirements, such as in form of odorant-saturated plastic polymer beads (see FIG. 1, 3a), liquids, gel or powder. Each cartridge may have at least one channel that contains at least one pipe. Each delivery output could have individually controllable air channels connected to scent cartridges. All channels and piping system should be made preferably of various non-porous and odourless material and non-absorbent material such as PTFE-F. The device may have at least one chemical substance container directing the flow with at least one chemical substance to a head space or to a nozzle comprising at least one smell output.

A various non-porous, non-absorbent and odourless material nozzle or smell output is in communication with at least one channel connected to at least one chemical substance container.

The cartridge may have a user-friendly slot in/out with a modular design solution from 1 unit to n-units. The device may have no cross-contamination between channels and requires perfect sealing, avoiding air flow drops or smell leakage.

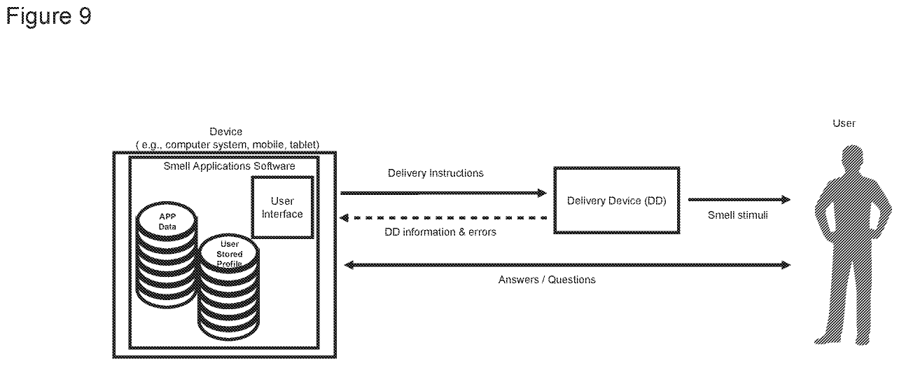

FIG. 9 shows a block diagram of the adaptive smell delivery system unit. The smell delivery device, user, user feedback, and adaptive system unit are shown. The communication between these units is also shown.

An adaptive system unit is provided which communicates with the smell delivery device (which is described above) via a communication unit configured to communicate with the smell delivery device. The adaptive system unit further comprises an input unit configured to receive instructions from a user. The adaptive system unit may process these user instructions to determine instructions to send to the smell delivery device regarding changes to the flow rate. Alternatively, the adaptive system unit may send the user instructions directly to the smell delivery device. The smell delivery device may then determine any changes required to the flow rate from the user instructions itself. Either way, the communication unit sends information to the smell delivery device (either in the form of the user instructions, or direct instructions to the smell delivery device).

The smell delivery device emits smell stimuli in accordance with the instructions determined from the user instructions. These stimuli are emitted in the vicinity of the user. The user then provides further feedback to the adaptive system unit as shown in FIG. 9. This feedback constitutes further user instructions. The process shown in FIG. 9 may therefore be iterative.

Additionally, any errors or other information may be sent by the smell delivery device to the adaptive system unit. These errors may trigger a further instruction, such as an instruction to shut down or stop the process.

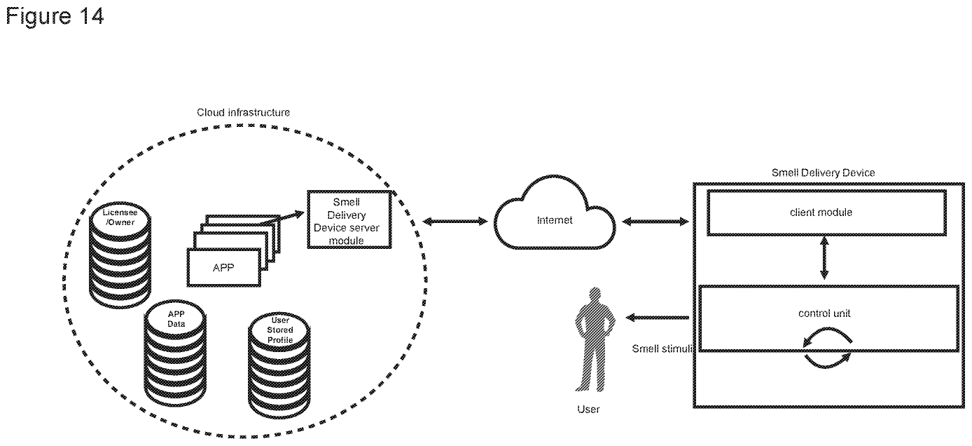

The adaptive system unit may be independent, as in FIG. 8, or incorporated into the smell delivery device, as shown in FIG. 14.

The type of adaptive system unit disclosed incorporates a smell application which adaptively controls and instructs the smell delivery through a system sensing and computing environmental, chemicals and perceptual factors.

FIG. 9 presents a block diagram of the overall adaptive system architecture which may integrate and store input from a user through an input unit relative to one or more smell applications (e.g., smell training, smell testing, audio-visual contents, Virtual Reality or Augmented Reality or Mixed Reality implementations) with one or more local and/or distributed delivery devices adapting the delivery parameters and in function of the user's active or indirect input through sensory information.

The overall system may comprise hardware components including the smell delivery device. The smell delivery device may be as described above. Alternatively, or in addition, the smell delivery device may be composed of at least a flow controller, optionally in the form of an electronic unit control, and one flow generator, such as a module of a pump-valve (or other forms of actuators), a canister with a delivery channel and at least one sensor (e.g., a sensor to determine flow rate through the delivery channel, an environmental sensor, or a user feedback device). The electronic control unit may contain a microcontroller or a microprocessor or a state-machine circuit able to interface with the electronic unit, analyse the signal from the at least one sensor and control the signal to the valve.

The control unit may drive the sensing and modulating of the various delivery parameters (e.g. frequency, intensity, activation channels, user distance adjustment etc.). The flow generator system and the solenoid valves may have the advantage of being able to deliver with a consistent and stable air flow across one or several channels replicating identical stimuli over time, adjustable to user's viabilities, chemical substance characteristics and environmental factors as such relative location. The adaptability may be determined by users' information and inputs and the synchronized sensors inputs. The sensor inputs may represent a security control feedback of behaviour of the overall system, allowing the detection of errors or malfunctions.

The device may be a portable version by inclusion of a battery and power management technology or non-portable with an external power supply. The external power supply may for example be 12V. The different sensors integrated in the system may have different power consumptions, for example the air flow sensors may have continuous power consumption for flow sensors, which may be less than 10 mW. The total power consumption of the device is preferably in the range of 1-2 A at 12V (12-24 W), however, the system may have low-powered electronics in other embodiments, such as those designed for the portable use.

As shown in other Figures (such as FIG. 7) the smell delivery system may include a power input, a connectivity input and in some embodiments a display, such as an LED or LCD display. A display may be used to communicate the basic status of the smell delivery device.

To explain the functionalities and methods of the adaptive system, FIG. 9 presents a block diagram of the overall components. The user interface, the APP data and the user stored profile are components common to the Smell Application Software (named APP), while each APP may have a proprietary coordinator component with the application logic in it.

In the majority of the possible applications and the APP implementations a perceptual profile of the users may be inferred by the device. In this particular example a smell stimulus may be tailored to user's perception threshold level (e.g., in smell training or a smell test), the application logic coordinates the delivery of smell stimuli by the delivery device and a separate interaction with the user by questions and answers related to that smell stimuli. The APP may be interacting with a user through an input unit or by receiving data indicative of user's biofeedback (such as heartrate, skin conductance etc.), presenting the questions and answers graphically or in any other modality to capture the users' response to perception features and personal information. This is a dynamic process where the questions of the user are adapted to the user's answers which are translated by the coordinator in delivery instructions. The control unit inside the device may adjust the delivery stimuli to the dynamic process expressed in delivery instructions. Likewise, the control unit integrates the input for the sensors and sends them back to the APP relative to the state-machine feedback (e.g. errors). The control unit may coordinate the mechanical and software components to adjust the delivery parameters with the algorithm driving the sensors.

The overall APP may be located in a device (e.g., computer system, mobile, tablet computer), as presented in FIG. 9 or be deployed in a cloud-infrastructure, as presented in FIGS. 10, 11, 13, and 14. In the case of a cloud-based APP, the information provided by the user as well as their profiles for later use with other APPs, could be stored in the cloud with user access or for agreed third party use. For instance, the digital records for smell training performances and preference or smell testing scores could be accessible by single users or healthcare professionals or relevant stakeholders (e.g., GPs, Hospitals, Clinics, etc.). The smell delivery device may contain multiple connectivity mechanisms, including Bluetooth, ethernet, USB, Wi-Fi, RF technologies to directly or indirectly connect with external devices and systems.

FIG. 10 shows a block diagram of adaptive smell delivery system unit embedded in a cloud-based configuration. The adaptive system unit in this example is therefore a virtual machine, rather than a real one. The user provides their instructions through answering questions posed to them. This feedback is communicated to the cloud infrastructure. The cloud infrastructure then communicates with the smell delivery device. The cloud infrastructure may perform a method, using the user's feedback, or perception, or biofeedback, to determine instructions to provide to the smell delivery device. The smell delivery device may send error indications, or feedback to the cloud infrastructure.

In this embodiment the user may provide feedback into an external device, such as a tablet, that is connected to the cloud infrastructure.

To support multiple use applications the delivery device may be integrated in a variety of settings, interacting with other devices (computers, smartphones, tables, PDA, haptic device, VR headset, etc.) and being guided by different software applications that may be deployed in one or more of those devices. In order to communicate with those devices, the smell delivery device may contain multiple connectivity mechanisms, including Bluetooth, ethernet, USB, Wi-Fi, and/or RF technologies. One or multiple smell delivery device may be used in the same applications or by different applications run synchronously or not, according to all the alternative system configurations as below presented in detail.

Depending on the use case applications, the smell delivery device may advantageously, for instance, have a low latency integration with other devices presented in the same physical space or there may be distributed delivery devices that need to update a remote computer in the cloud with collected information from users in multiple locations.

According to a further aspect of the present disclosure related to the adaptive unit there are various types of embodiment integration configurations provided. We describe as examples here possible integration settings for the system. The smell delivery device connects directly (via USB port for serial communication, Bluetooth, Wi-fi, RF technologies, etc.) to other general devices (computer, mobile, tablet, etc.) located in the same physical space, as presented in FIG. 8.

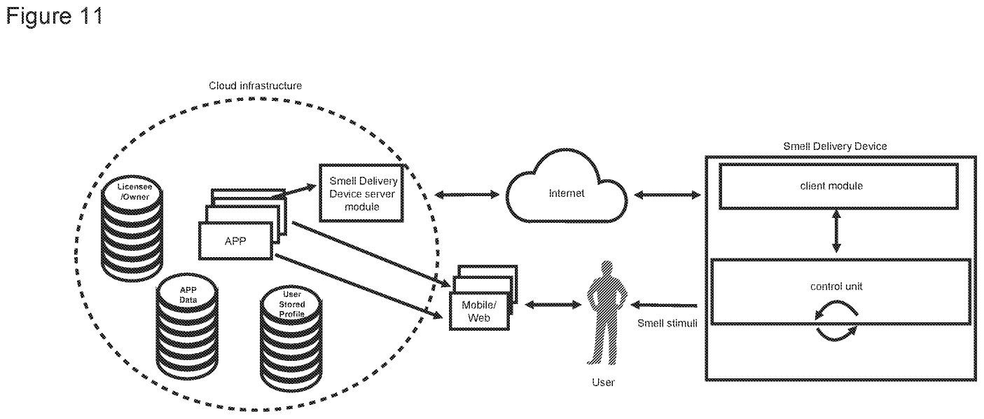

FIG. 11 shows a system configuration where the delivery device is in a different physical space and connected through the Internet (using a client/server architecture) to a remote computer, as in a cloud infrastructure, or a physical machine. This example of configuration could be applicable to a numerous Internet-of-Things applications.

The smell delivery device connects through Internet (using a client/server architecture) to a remote computer, as in the cloud or in-premises. That remote computer may connect to a second device (computer, mobile, tablet, etc.) co-located with the smell delivery device, which may be used as the user interface. FIG. 11 presents an example of a cloud-based infrastructure with an APP), that may use cloud-storage to save information relative to user stored profile data, APP data, usage license, etc. The APP may communicate with the smell delivery device through a communication module using a proprietary protocol. In the smell delivery device a counter-part to this module, DD client, could take care of the communication with the remote computer and could interact with the control unit (FIG. 11). The APP and the DD client are two modules to interpret communication, one sitting in the sever and one in the smell delivery device to allow interchange protocols, in order to enable communication between the server and the smell delivery device. This example of configuration could be applicable to a numerous Internet-of-Things (IoT) applications or devices.

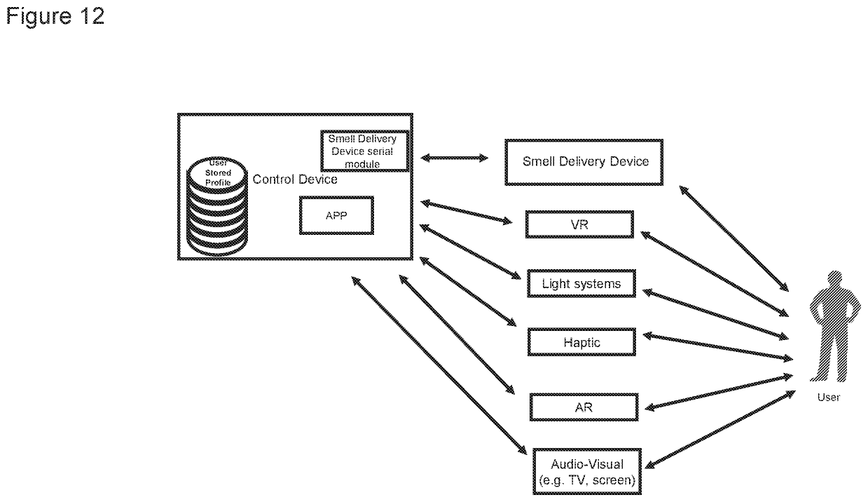

FIG. 12 shows a further system configuration where the delivery device connects directly (via USB port for serial communication, Bluetooth, Wi-Fi, RF technologies, etc.) to a device used as orchestrator with other devices in the same physical location (like audio-visual screen, VR sets, haptic devices, light systems, etc.) to achieve a low latency integration and deliver a tight coordinated multisensory experience.

The smell delivery device connects directly (via USB port for serial communication, Bluetooth, Wi-fi, RF technologies, etc) to a central device used to control other device. For example, the central device may interact with and control other devices in the same physical location (like audio-visual screen, VR sets, haptic devices, light systems, etc.) to achieve a low latency integration and deliver a coordinated multisensory experience. FIG. 12 represents a diagram block with an example of the main components.

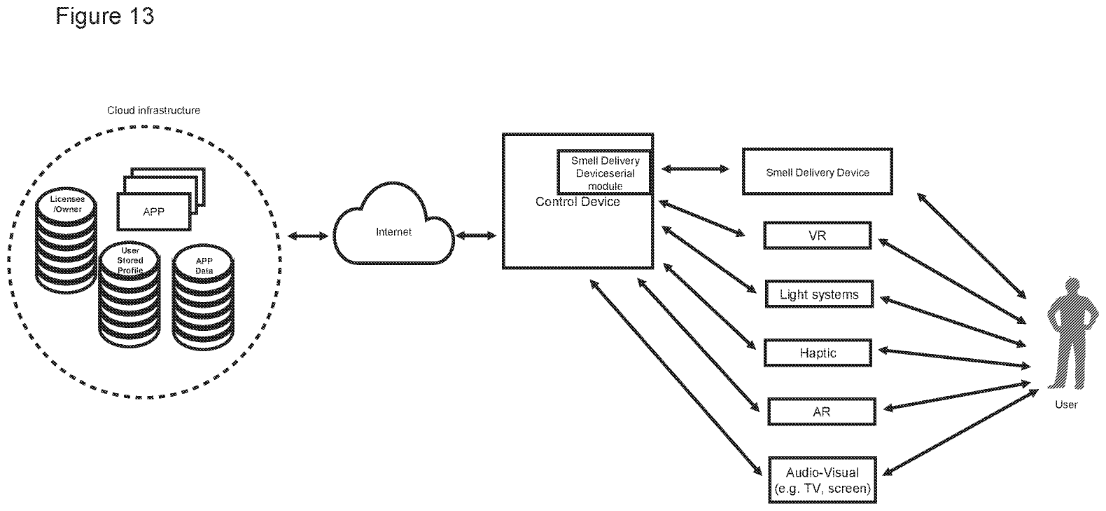

FIG. 13 shows a further system configuration where the delivery device, as shown in FIG. 10, is used differently in such a way that the central device connects to a remote server through the Internet (to deliver information, obtain instructions, etc.).

This configuration is the same as the previous configuration presented in FIG. 12 above with the difference that the central device connects to a remote server through the Internet (to deliver information, obtain instructions, etc.). The components are shown in FIG. 13. This example of configuration could be applicable to a numerous Internet-of-Things (IoT) applications or devices.

FIG. 14 presents a possible system configuration where the delivery device is connected through the Internet to a remote computer that may be provided by the cloud or by in-premise servers to deliver a general experience that does not require input from the user.

The smell delivery device connects through the Internet to a remote computer that can live in the cloud or in-premise servers to deliver a general experience that does not require input from the user or retrieval information from stored user profile or APP information. FIG. 14 represents a diagram block with an example of the main components.

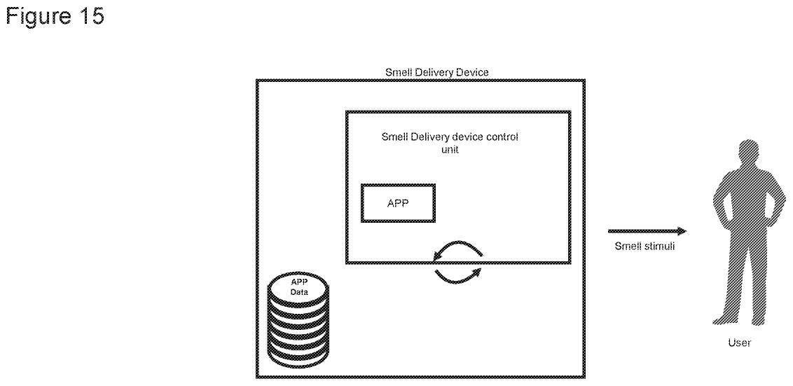

FIG. 15 shows a block diagram of a further system configuration where the delivery device functions in isolation based on an application running on its own automated delivery device control unit. Therefore, in FIG. 15 the smell delivery device does not receive any feedback from the user, and instead runs a pre-set program. This may be particularly useful in use cases in the entertainment industry. For example, virtual reality devices may encompass the smell delivery device in order for all of the human senses to be stimulated by the virtual reality device. Therefore, the smell delivered by the virtual reality device may be dependent on the virtual reality shown to the user, rather than being dependent upon user feedback.

Therefore, in this embodiment the smell delivery device functions in isolation based on an application running on its own microprocessor. This could be a predefined set delivery experience that does not require input and could retrieve information from a stored user profile or APP information locally-stored in the smell delivery device flow controller, or in another control unit in communication with the smell delivery device. This simple configuration is presented in FIG. 15.

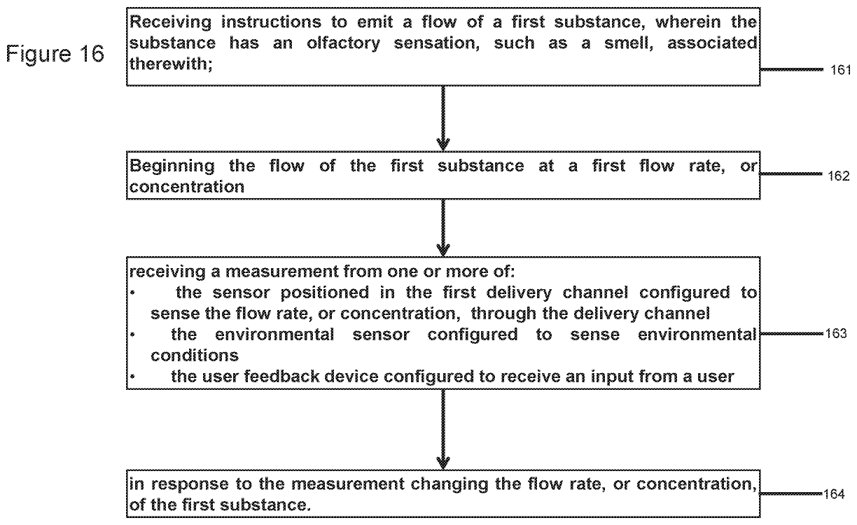

FIG. 16 shows a method of delivering a smell from the smell delivery device. The method comprises the first step 161 receiving instructions to emit a flow of a first substance, wherein the substance has an olfactory output, such as a smell, associated therewith. The second step 162 comprises beginning the flow of the first substance at a first flow rate, or concentration. The method then 163 comprises receiving a measurement from one or more of: the sensor positioned in the first delivery channel configured to sense the flow rate, or concentration, through the delivery channel, the environmental sensor configured to sense environmental, and/or the user feedback device configured to receive an input from a user. The final step 164 in FIG. 16 is in response to the measurement changing the flow rate, or concentration, of the first substance.

The adaptive system unit is configured to receive instructions from a user or biofeedback measure and then process the instructions or feedback to determine a flow instruction. The flow instruction is to modulate the flow generated by the flow generator. This flow instruction is then sent to the smell delivery device. The smell delivery device receives the flow instruction and in response modulate the flow rate, or concentration of the first substance, accordingly.

For example, the environmental sensor of the smell delivery device may detect the temperature, humidity, or pressure, of the local environment. This may affect a user perception of smell. For example, in the event that the temperature is very cold (for example close to or below 0 degrees Celsius) the volatility of molecules, including substances stored in the canisters may be reduced. This may reduce the smell associated by the substance, and so a higher flow rate, or a higher concentration of a substance may be required. Therefore, the smell delivery device may in response modulate the flow to increase the flow rate, or concentration of the first substance. Similarly, if humidity is high, the user may experience a musty smell. Therefore, to overcome this effect the flow rate may have to be increased, or another substance also emitted to mask the odour associated with the humidity.

If the sensor in the delivery channel senses that the flow rate/concentration is different to that required then it will feed back to the flow controller that the flow rate, or concentration should be modulated.

User feedback will also lead to a response. This is dependent on the feedback provided by the user, and the questions that may have been asked to the user. This will depend on the specific application, or test that the user is undergoing.

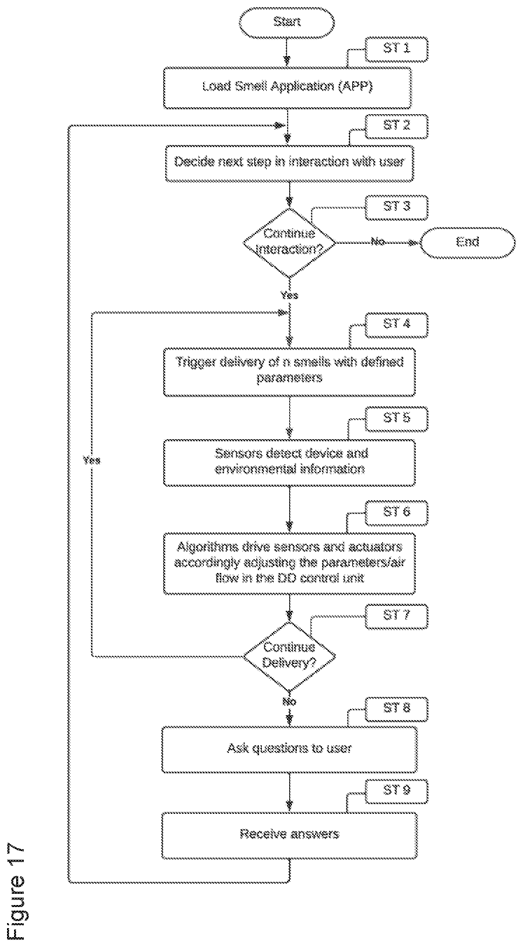

FIG. 17 shows an example of a process of a smell delivery method implemented according to an embodiment integration configuration setting, based on the example of infrastructure configurations as presented in FIGS. 9, 10, 11 and 12.

In the flow chart an example of a possible smell application (APP) is described. In ST1, the APP is loaded and launched by a computing device, which may determine the next step of interaction with the user (or users) (ST2) initiating a loop of interactions between the user and the computing device for which conditions to continue interacting are evaluated in ST3. The smell delivery will be triggered with defined parameters (ST4) and following that the sensing of the environment and the delivery system itself by the smell delivery device (ST5) will allow the algorithm to adjust its delivery parameters to control the actuators in order to modify the airflow as required (ST6).

The next step is a condition to generate a feedback loop in which the process of other iteration of smells delivery/sensing/adjustment of delivery parameters can be triggered until the goal of delivery is fulfilled (ST7). The whole feedback loop process, from ST4 to ST7, may be performed by the DD's control unit.

After delivering smell the interaction with the user is based on questions presented to the user (ST8) and their answers supplied to the computing device (ST9), which brings the flow back to defining based on this new information how the program will continue, by returning to ST2. From this point the entire cycle might repeat, or based on the decision made in ST2, decide not to continue the interaction with the user and finish the execution of the application.

Note that in the example of FIG. 17 the APP may be locally in device (e.g., computer system, mobile, tablet) as shown in FIG. 9 or in an orchestrator as in FIGS. 12 and 13, or run in a cloud infrastructure as in FIGS. 10 and 11.

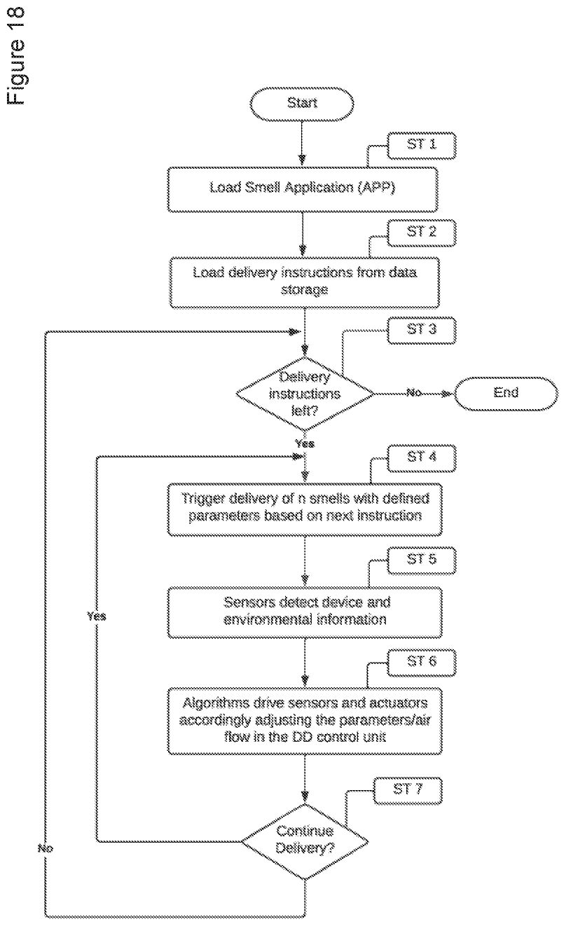

FIG. 18 shows a flow chart illustrating an exemplary flow process of a smell delivery method implemented according to an embodiment integration configuration setting, based on the example of infrastructure configurations as in FIGS. 14 and 15.

In the flow chart an example of a possible smell application (APP) is described. In ST1, the APP is loaded and launched by a computing device, which may load the next step of delivery instructions (ST2) from a data storage, initiating a loop of interactions for which the condition to continue operating is based on delivery instructions left to be executed (ST3). The smell delivery will be triggered with defined parameters based on the next delivery instruction (ST4) and following that the sensing of information from the environment and the delivery system itself (ST5) will allow the algorithm to adjust its delivery parameters to control the actuators component in order to modify the airflow as required (ST6).

The next step is a condition to generate a feedback loop in which the process of other iteration of smells delivery/sensing/adjustment of delivery parameters can be triggered until the goal of that particular delivery instruction is fulfilled (ST7). The whole feedback loop process, from ST4 to ST7, may be performed by the DD control unit.

After delivering smell the flow returns to ST3, where it will check for further instructions. From this point the entire cycle might repeat, or no, more delivery instructions left to finish the execution of the application.

Note that in the example of FIG. 18 the APP may be locally stored in the smell delivery device as shown in FIG. 15 or run in a cloud infrastructure as in FIG. 14.

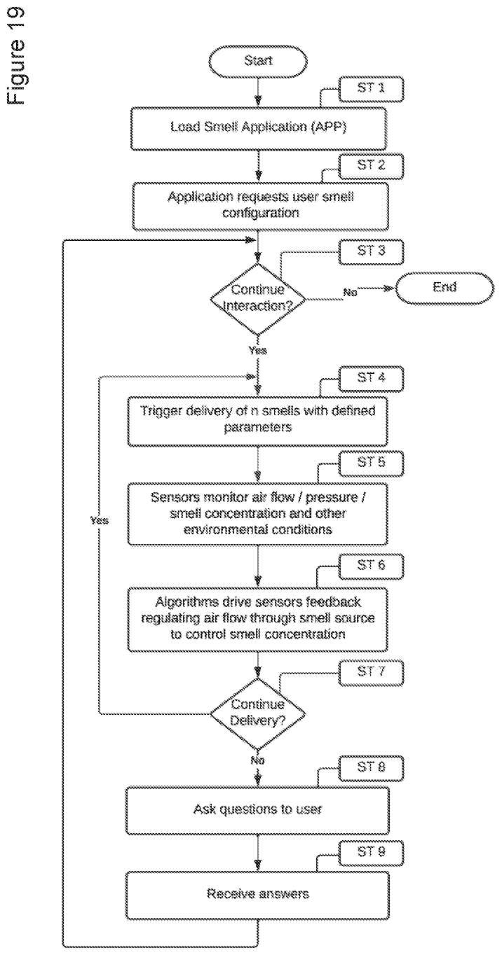

FIG. 19 shows a flow chart of an exemplary process of a smell delivery method implemented according to an embodiment integration configuration setting, which may be based on the example of infrastructure configurations as presented in FIGS. 9, 10, 11 and 12.

In the flow chart an example of a possible smell application (APP) is described. In ST1, the APP is loaded and launched by a computing device, which may request specifically a user smell configuration (ST2) initiating a loop of interactions for which conditions to continue interacting are evaluated in ST3. The smell delivery device will be triggered with defined parameters (ST4) and following that the sensing of the environment and the delivery system itself, specifically air flow, pressure, smell concentration (ST5) will allow the algorithm in driving the sensor feedback to regulate the air flow of the delivery through smell source to control scent concentration (ST6).

The next step is a condition to generate a feedback loop in which the process of other iteration of smells delivery/sensing/adjustment of delivery parameters can be triggered until the goal of delivery is fulfilled (ST7). The whole feedback loop process, from ST4 to ST7, may be performed by the DD's control unit.

After delivering smell the interaction with the user is based on questions (ST8) and their answer (ST9), which brings the flow back to defining based on this new information how the program will continue, by returning to ST3. From this point the entire cycle might repeat, or based on the decision made in ST3, decide not to continue the interaction with the user and finish the execution of the application.

Note that in this example as in FIG. 19 the APP may be locally in device (e.g., computer system, mobile, tablet) as shown in FIG. 9 or in an orchestrator as in FIGS. 12 and 13, or run in a cloud infrastructure as in FIGS. 10 and 11.

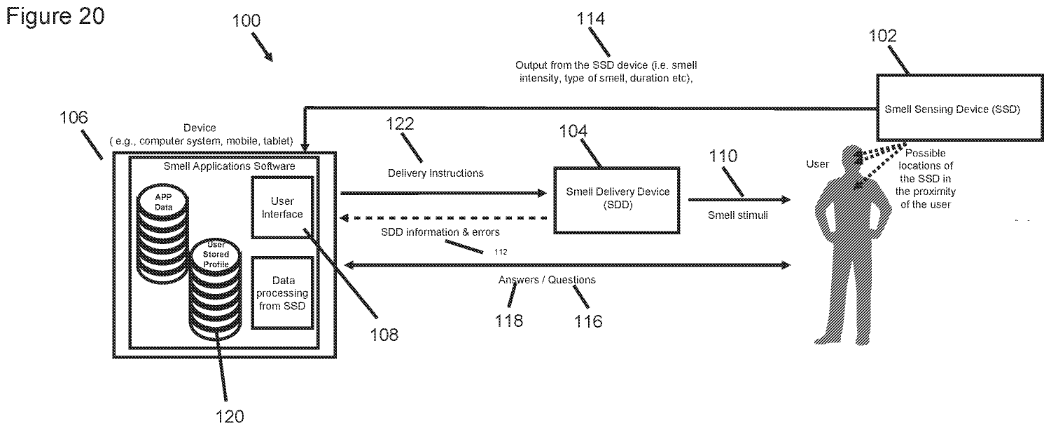

FIG. 20 shows a block diagram of an example of an adaptive smell delivery system unit together with a smell sensing device. This figure is similar to FIG. 9 but an additional smell sensing device is present. The system of FIG. 20 corresponds to a smell aiding system 100 according to one embodiment. The system of FIG. 20 may also be referred to a smell sensing system and thus the system also corresponds to a smell sensing system 100 according to one embodiment. The smell aiding system 100 comprises a smell sensing device 102. The independent unit of a smell sensing device 102 is ideally placed in the proximity of the user. The smell sensing device 102 could be placed for example around the nose or face or on the clothes of the user, for example by attachment via a clip. The reason for this is for the smell sensing device 102 to give an accurate representation of the concentration or intensity of the smell or the smell duration that can be live (or non-live) correlated with the perceptual olfactory sensation of the user. The smell aiding system 100 also comprises a smell delivery device 104. Communication channels between these units are also shown. The smell aiding system 100 also comprises a computing device 106 having a user interface 108. In other words, the smell sensing system 100 comprises a smell sensing device 102 and a smell delivery device 104.

The smell delivery device 102 comprises an array of gas sensors. The array includes a plurality of MOX sensors and a PID sensor for calibration. The gas sensors are configured to generate sensor information in response to an olfactory output 110. In FIG. 20, the olfactory output 110 is emitted by the smell delivery device 104. The olfactory output results in smell stimuli which should be detectable by the nose of the user (assuming their sense of smell is not impaired). In other examples, the olfactory output 110 is natural or emitted by a smell source other than a smell delivery device 104 so the system 100 can be provided without a smell delivery device 104. The smell delivery device 104 outputs an olfactory output 110 such as a lavender smell (or a combination of smells, perhaps at different intensities). The olfactory output 110 (smell stimuli) is then detected by the gas sensors of the smell sensing device 102. The olfactory output 110 may also be detected by the user, but in some cases the user may have an impaired sense of smell.

The smell sensing device 102 is configured to output sensor information 114 to the computing device 106. The sensor information 114 comprises the output of the gas sensors of the smell sensing device 102. The sensor information 114 may comprise smell intensity, a type of smell, a duration of smell, and/or an identification of the smell. In this example, the smell sensing device 102 has a wireless connection to the computing device 106.

The computing device 106 may be a computer system, a mobile, or a tablet. For example, the computing device 106 may be a smart device such as a smartphone or tablet. In this embodiment, the computing device 106 is local. In other words, the computing device 106 is local to the user so the user can interact with it. The computing device 106 may also interact with devices such as the smell delivery device 104 and the smell sensing device 102 locally, in this case via a wireless connection but in other cases directly via wires. The computing device 106 has a user interface 108. The user interface 108 forms a user output device. The user interface 108 can output an identification of a smell associated with the olfactory output 110. For example, the user interface 108 can output information 118 to the user, for example through a screen of the computing device 106. The user interface 108 can also receive user input information 116 such as indicating a perception of the smell related to the olfactory output 110 or other questions. The user interface 108 in this example is a touch screen, but in other examples it may be different such as a button which is separate from the user output which may be a display screen.

The computing device 106 also comprises a processor. In this embodiment, the computing device 106 runs software which performs processing. The Smell Application Software (named APP) previously described in FIG. 9 receives the sensor information 114 from the smell sensing device 102. The computing device 106 therefore can contain useful feedback (such as live information) from the smell sensing device 102. The sensor information 114 may include the intensity of smell, the smell pulse duration, the type of smell, the channel selection, or may contain a reading of the background smell of the user or the environment around the user (baseline). Dynamic information, such as smell signal variation in time or sequence of smell pulses could also be provided by the smell sensing device 102 and used by the computing device 106. Accordingly, the computing device 106 acts as the processor as disclosed herein. The computing device 106 can process the sensor information 114 and can further output an identification of the smell to the user via the user interface 108. In other examples, the processor may be separate from the user interface 108.

The sensor information 114 from the smell sensing device 102 is further correlated with delivery information 112 which is data or information generated by the smell delivery device such as smell delivery flow rate, channel selection, pump pressure, electro-valve opening. The delivery information 112 is received from the smell delivery device 104. This may indicate what canisters are loaded, which delivery channels are active, which valves are open, and the flow rate through each delivery channel. The correlation can determine that the smell delivery device 104 has worked correctly as the expected olfactory output 110 has been released at the expected intensity, as confirmed by the output of the smell sensing device 102 identified in the sensor information 114.

The computing device 106 can further correlate the sensor information 114 with environmental information from an environmental sensor such as temperature, humidity, or ambient pressure. This is received from an environmental sensor which in this example is located on the smell sensing device 102. The environmental information may therefore be supplied alongside the sensor information 114 in this case and can be transmitted to the computing device 106. In other examples, the environmental sensors may be located elsewhere such as on the smell delivery device 104 or elsewhere. As it is known that parameters such as temperature and humidity affect a sense of smell, information identifying temperature or humidity may be used to determine an environmental effect on the olfactory output 110. For example, the information related to the olfactory output 110 from the sensor information 114 may be compensated based on the environmental information.

The computing device 106 can further correlate or process the sensor information (and optionally together with the delivery information 112 and/or the environmental information) with user information which can be further information or data from a user feedback device configured to receive an input from a user, wherein the input from the user indicates a parameter of the user's perception of the olfactory output. In this example, the user information 116 is received from the user interface 108 of the computing device 106 where the computing device 106 acts as a user input unit of a user feedback device. The user inputs information such as their perception of a smell, for example indicating whether they can detect the smell, their perceived intensity, and/or their perception of what smell it is. For example, the user may input that they detect a strong odour of lavender, in this case.

In other embodiments, this further correlation of the sensor information 114 with delivery information 112, environmental information, and/or user information 116 is not necessary and the delivery information 112, environmental information, and/or user input information 116 are not required.

The processor of the computing device 106 can then correlate the sensor information 114 with the delivery information 112, environmental information, and/or user information 116 to identify the smell of the olfactory output 110. For example, the sensor information 114 from the smell sensing device 102 can be processed and from this the processor can identify a smell from the olfactory output 110 (for example a lavender smell in this case). This may be based on algorithms such as neural networks (trained on test data) analysing the signal of the gas sensors. The array of gas sensors can be used together to detect different VOCs and calibrate each other. In combination, an accurate determination of the olfactory output 110 can be provided.

The analysis is compensated for effects of the environment, by processing the environmental information, e.g. the temperature and humidity, to compensate for effects on the olfactory output 110. For example, the sensor information 114 may be adjusted when the temperature or humidity is above a certain threshold, or adjusted using a formula dependent on temperature or humidity. The computing device 106 also has a user stored profile 120 of user information which can also be used. This can indicate user preferences relating to desired output, or historical information as to impairments to sense of smell. This can be used to adjust an output through the user interface 108 of the computing device 106, for example alerting the user of an identification of certain smells at a lower intensity to other smells where the user perception is lower for that smell compared to others. The user can also input information 116 through the user interface 108 of the computing device 106 which thus acts as a user feedback device. The user interface 108 can also output answers 118 to questions 116 from the user or other outputs 118 such as an identification of smell or other feedback. User information input to the computing device 106 can then be stored in the user stored profile 120.

Once the computing device 106 has identified the smell, it can then output the identification via the user interface 108 (user output device) in the form of an output 118. The identification is a representation of the olfactory output 110. In this embodiment, the identification is an image of an object associated with the smell, in particular the object that produces the smell. In this case, the computing device 106 outputs an image of lavender via the user interface 108 so that the screen displays an image of lavender. The user can then see this and understand that the smell is lavender. In cases whether the user smell is impaired, this can aid the user or confirm their sense where it is weak.

The computing device 106 also sends delivery instructions 122 to the smell delivery device 104. In this case, these adjust the olfactory output 110 by changing the smell or intensity or combination of smells. The adjustment of the olfactory output 110 can be based on the analysis of the computing device 106, for example considering the sensor information 114 of the smell sensing device 102 or other sensors. In one example, based on the sensor information 114 of the smell sensing device 102 indicating the intensity of the olfactory output 110 (for example, the sensor information 114 containing a signal such as a voltage indicating an intensity), the computing device 106 determines that the intensity of the olfactory output 110 is too low. Based on this, the computing device 106 instructs the smell delivery device 104 to increase the intensity. This can be used for smell testing and training. The smell delivery device 104 in this embodiment has multiple delivery channels for receiving a substance from a canister and an output component through which the substance can be emitted. The substance can produce the olfactory output 110 to be detected by the smell sensing device 102. The smell delivery device 104 also has airflow generating elements which generate airflow to transport the substance from the canister to the output component. In some examples, the smell delivery device 104 is the smell delivery device of other embodiments such as in FIGS. 1 to 15. In this example, there is also a flow controller to adjust the flow rate of the substance to change the intensity of the olfactory output, but this need not be provided in some embodiments. Different delivery channels can be used for different smells, and these may be combined in varying concentrations through flow controllers or valves in each delivery channel to result in a combined olfactory output. In other embodiments, the smell delivery device 104 is not required, and the system 100 may be implemented with other smell sources such as natural smells.

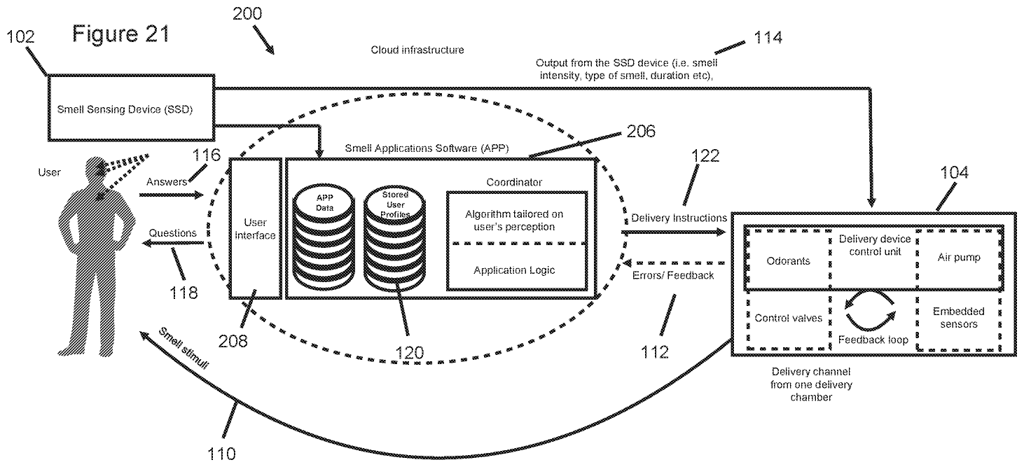

FIG. 21 shows a block diagram of an example of an adaptive smell delivery system unit and an independent unit of smell sensing device in a possible cloud-based configuration. In particular, FIG. 21 shows another embodiment of the smell aiding system 200 which is similar to the smell aiding system 100 except where set out below. The system of FIG. 21 may also be referred to as a smell sensing system 200. FIG. 21 is similar to FIG. 20, but instead of the processing being performed on a computing device 106 which is local to the user, the processing is performed remotely in a cloud infrastructure. This figure is similar to FIG. 10, but it shows the addition of a smell sensing device 102 and its communication channels to the computing device 206 in the cloud. The cloud should preferably be understood to mean computing resources (such as processors and servers, data storage, data access, and/or software) available via a network such as the internet. Thus, cloud computing can refer to storing and accessing data and programs over the internet instead of on a local computing device.

In this embodiment, the computing device 206 is a remote computing device and is arranged in the cloud infrastructure. In other examples, more than one computing device is provided where the processing is shared across multiple devices or resources in the cloud. In some examples, part of the processing can be performed in the cloud, while part can be performed locally by a local computing device such as computing device 106. The computing device 206 interacts with the smell sensing device 102 and the smell delivery device 104 in a similar manner, but over a remote connection such as over the internet. In this embodiment, the system 100 includes a user interface 208 which is in communication with the computing device 206 in the cloud. In this embodiment, the user interface 208 is a smart device such as a smartphone. The user can interact with the user interface 208 of the smart device to communicate with the computing device 206 in the cloud.

The smell sensing device 102 may comprise a Bluetooth component or a wireless transmitter and can be connected wirelessly to the computing device 206 or directly to the wireless network. The sensor information 114 from the smell sensing device 102 is further processed in the computing device 206 in the cloud to more accurately predict and differentiate between different categories of smells and their intensities. Machine learning, neural networks or more advanced artificial intelligence (AI) algorithms may be used on the computing device 206 to increase selectivity and sensitivity to smell. As the computing power can be higher when using a cloud service compared to a local device, more complex algorithms such as more powerful AI algorithms can be used. The computing device 206 can also communicate with the user interface 208 for outputting information 118 to a user and receiving input 116 from the user. This allows for the user to input their perception to a smell 116 and to receive an output 118 identifying the smell. The user interface 208 thus acts as a user output device and also as a user feedback device having a user input unit.

In other words, the system 200 can be operated in a similar manner to the system 100, except that the processing is performed in the cloud.

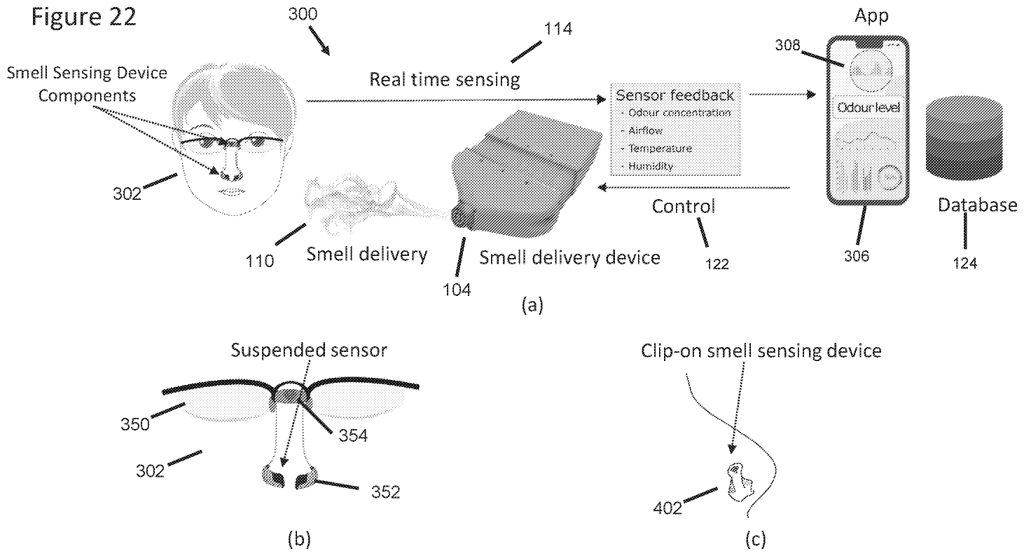

FIG. 22A shows a schematic representation of an example of the various components involved in smell training or smell testing. FIG. 22A shows an example embodiment of a smell aiding system 300, which is similar to the smell aiding system 100 or 200, except as set out below. The system of FIG. 22 may also be referred to as a smell sensing system 300. The system 300 has a smell sensing device 302, a smell delivery device 104, and a smart device 306 for processing. The smart device 306 is a computing device and has a user interface 308. The system 300 is similar to FIG. 20 or 21. The smart device 306 is a smartphone having an app which can output information such as an identification of the smell and odour levels. In other examples, the smart device 306 may be a tablet or other smart device. The app can also receive user input such as a perception of a smell. The smell delivery device 104 delivers a smell by emitting an olfactory output 110 towards the user.

The smell sensing device 302 is attached to the user so that it is close to the nose of the user. Here the position of the smell sensing device 302 attached to the frame of glasses is specifically shown. The odour is delivered by the smell delivery device 104 via volatile organic compounds. The VOCs diffuse through the medium (air) and part of them reach the user. The intensity of the smell is given by the concentration of the VOCs that reach the user. This in turn is a function of the flow rate through which the smell is delivered in each channel. The smell sensing device 302 detects the concentration of the smell, the type of smell, the smell duration (in the proximity of the user), the background smell and also ambient parameters such as humidity and temperature. In this example, the smell delivery device 302 comprises gas sensors and environmental sensors.

The smell sensing device 302 provides a live feedback to the smart device 306. The smell delivery device 104 has a wireless transmitter for communicating with the smart device 306 by Bluetooth or other wireless protocol such as Wi-Fi. The sensor information 114 sent from the smell sensing device 302 to the smart device 306 contains a signal of the sensor response to the olfactory output 110 which indicates an intensity of the smell and the data from the smell sensing device 302 may further contain environmental information from environmental sensors indicating, for example, temperature and humidity.

The information is correlated with a database 124 by the smart device 306 and the feedback olfactory perception from the user. The user feedback can be input via the user interface 308 to the smart device 306. Where the user indicates that they perceive the smell (or indicate a relative intensity), the sensor information 114 can be used to verify this. The database 124 can be used by the smart device 306 to look up historical data such as background olfactory output patterns or historical user data. Thus, the sensor information 114 may be correlated with user information and user input information. In the event that the user indicates that they cannot detect the olfactory output 110 (e.g. lavender smell) emitted by the smell delivery device 104, but the smell sensing device 102 indicates a high intensity of, in this case, lavender, via the sensor information 114, then it is confirmed that the system 300 is working correctly, indicating an impairment to the user's sense of smell. By processing the historical user information, the smart device 306 can determine that the user has historically had difficulty smelling lavender. This information can be output to the user via the user output device, which in this case is provided by the user interface 308 of the smart device 306. In this embodiment, the user output device is the same device (smart device 306) as the processor and the user input device, but in other examples they may be provided separately. This information can also be fed back to the smell delivery device 104 through control signals 122.

The smart device 306 also communicates with the smell delivery device 104 to control channel selection, flow rate, smell intensity and other parameters. The smart device 306 can instruct the smell delivery device 104 to adjust the olfactory output 110. If, as here, the user has indicated they cannot detect the smell, the smart device 306 can instruct the smell delivery device 104 to increase the intensity such as by increasing flow rate to see if the user can detect the higher intensity. The smart device 306 may communicate with the smell delivery device 104 wirelessly such as over a wireless network or by Bluetooth. The user information can indicate a level at which the user can detect the lavender smell, so the intensity can be changed to confirm this level and detect any change in the user's ability to smell.

The smart device 306 is the processor in this example and may receive the sensor information 114 and delivery information, and send instructions 122 to adjust the delivery of the olfactory output 110. The smart device 306 also performs the processing and outputs the identification or validation through the user interface 308. In other words, the processing is performed locally by the smart device 306. In other examples, the smart device 306 can act as a communication device and the processing can be performed on another device such as in the cloud. Thus, the smart device 306 can receive the information, but forward this to a remote computing device in the cloud. In other examples, the system 300 may include processing power in the cloud network.

FIG. 22B shows part of FIG. 22A, specifically elements of an example smell sensing device 302 of FIG. 22A attached to a specially designed pair of glasses 350. These glasses 350 could be with prescription lenses, clear lenses, dummy lenses, sunglasses lenses, or no lenses at all. In this example, a wireless transmitter 354 is attached to the frame of the glasses 350 while a unit comprising the gas sensors 352 (and optionally other sensors such as environmental sensors) from the smell sensing device 302 are clipped-on the nose, but in other cases could be connected to the frame of the glasses 350 (not shown). The smell sensing device 302 can be attachable to the glasses 350 so as to be retrofitted or may be provided integral with the glasses 350. The clips holding the gas sensors 352 may be attached to the glasses 350 or not.

FIG. 22C shows a simplified view of an example clip-on device of a smell sensing device 402. The position of the smell delivery device 402 close to the nose of the user is preferable, as this will give a more accurate correlation with the feedback provided by the user concerning his/her perceptual sensation of smell. In this embodiment, the smell sensing device 402 comprises a clip for attaching to the user's nose. In this example, a wireless transmitter is also provided on the clip-on device, rather than on the frame of glasses. To reduce weight, in some examples there is not a wireless transmitter but a data storage for storing the sensor information. This can then be plugged into a computing device to extract the sensor information for use by the processing device. The smell sensing device 402 allows the gas sensor to be located in proximity to the user's nose, meaning that the results of the sensor are indicative of the smell the user would perceive, and are more accurate than being further away. This can be used to aid or replace the user's sense of smell.

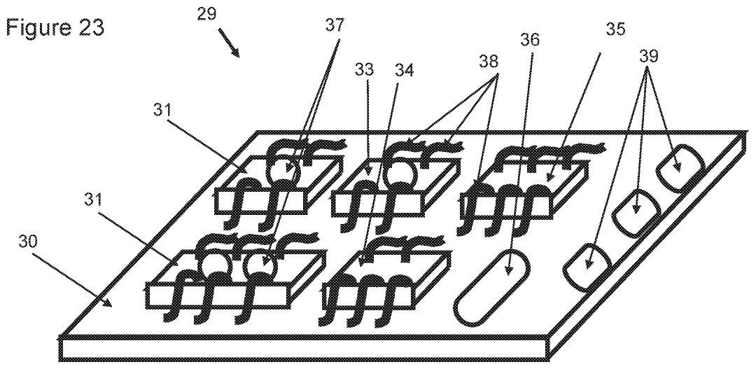

FIG. 23 shows a 3D schematic representation of an example of a smell sensing device 29, comprising a printed circuit board (PCB) 30, gas sensors in the form of multi-sensing volatile organic compound sensors 31, 32, environmental sensor 33, an ASIC drive and read-out circuit 34, a wireless transmitter/Bluetooth 35, battery 36, and interface ports 39. Openings 37 in packages or lids to allow smell to diffuse into the sensing elements of the volatile organic compound sensors are provided. Openings could also be provided for access to ambient conditions (e.g. humidity level). Metal leads 38 to interconnect circuits on the PCB are also schematically shown. The VOC sensor 31 is a PID sensor while 32 is a MOX array of sensors. The PID sensor 31 provides an accurate and stable detection of the total VOCs (TVOCs) present and a reliable reading of the baseline (background smell). The selectivity is improved to give information about the type of smell by combination with the MOX sensors 32. The array of the MOX sensors 32 can differentiate between different types of smell. This is facilitated by software algorithms implemented in the local ASIC 34 or alternatively as part of the app software of the smart device processing the sensor information. The PID 31 is also used to calibrate the MOX sensor array 32. The environmental sensor 33 comprises a temperature and humidity sensor. The temperature and humidity sensor is used to help to compensate for the parasitic effects of temperature and humidity on the output of the VOC sensors 31, 32. They can also be used as part of the ambient data and transferred as feedback to the smart device. The smell sensing device 29 may be used in the smell sensing device 102, 302, or 402.

While this figure shows an example of a simple, schematic implementation of a smell sensing device, other implementations based on different state-of-the-art assembly techniques are possible (not shown). The smell sensing device, could be in the form of a system in package (SIP) and could use techniques such as flip-chip, stack die, chip on board assembly, wafer level packaging etc. The VOC sensors could be for example mounted straight onto the chip of an ASIC rather than through a PCB, using a stack die technique. The humidity and temperature sensor may be co-packaged with the VOC sensor(s), to decrease the form factor and/or reduce the cost of the device.

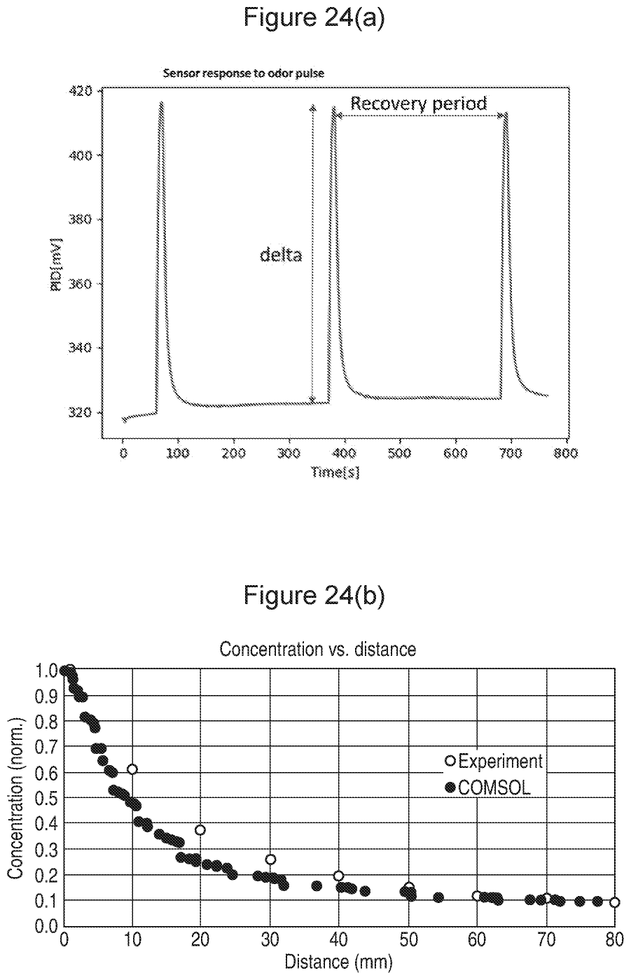

FIG. 24A shows the smell transient signal from a photoionization detector (PID) incorporated in an example of a smell sensing device. This figure shows a possible output of a smell delivery device function of time. The smell intensity, the smell duration and the baseline of the smell are shown. Between smell pulses a recovery period is present, where the smell reduces to that of the background smell. The signal from the PID sensor is shown as a voltage output. Here three identical pulses of smell delivered by the smell delivery device are present. A sequence of smell pulses with different intensities and different types of smell could be used as part of the procedures for smell testing, smell training, or smell immersive experience. This can form sensor information which can be processed by the processor using pattern-matching or other algorithms to identify the smell. For example, the signals can be compared to known signals in a database, or machine learning, neural networks, or AI methods can be implemented to identify the smell.

FIG. 24B shows the decay of the concentration of the smell as function of the distance between the smell delivery device and the smell sensing device. This figure clearly indicates that the intensity of the smell delivered by the smell delivery device and measured by the smell sensing device is highly dependent on the distance. The correlation is shown for both experimental data and simulation data using a software simulation package, in the example shown COMSOL, which is supplied by COMSOL Ltd, Cambridge, CB3 0DU, United Kingdom. The closer the user is to the smell delivery device, the stronger the olfactory perception. Placing the smell delivery device in the close proximity of the user (the user's nose) is preferable for a more accurate reading of the VOC concentration (smell signal). The smell intensity is also a function of the concentration of the chemicals in the cartridge and the flow rate through each of the channels of the smell delivery device. By use of a position sensor, the distance between the smell sensing device and the user's nose can be used to compensate the reading of the smell sensing device. In other words, if the distance is known, then the value of intensity of the gas sensor can be compensated based on the known correlation during calibration to provide a more accurate value of the intensity at the user's nose.

It will be appreciated from the discussion above that the embodiments shown in the Figures are merely exemplary, and include features which may be generalised, removed or replaced as described herein and as set out in the claims. With reference to the drawings in general, it will be appreciated that schematic functional block diagrams are used to indicate functionality of systems and apparatus described herein. For example, the functionality provided by the flow generator may in whole or in part be provided by the valves. In addition, the process functionality described may also be provided by devices which are supported by the adaptive system unit. It will be appreciated however that the functionality need not be divided in this way and should not be taken to imply any particular structure of hardware other than that described and claimed below. The function of one or more of the elements shown in the drawings may be further subdivided, and/or distributed throughout apparatus of the disclosure. In some embodiments the function of one or more elements shown in the drawings may be integrated into a single functional unit.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

In some examples, one or more memory elements can store data and/or program instructions used to implement the operations described herein. Embodiments of the disclosure provide tangible, non-transitory storage media comprising program instructions operable to program a processor to perform any one or more of the methods described and/or claimed herein and/or to provide data processing apparatus as described and/or claimed herein.

The sensors, flow controller and flow generator (and any of the activities and apparatus outlined herein) and the smell sensing device any of their constituent parts may contain or may be implemented with fixed logic such as assemblies of logic gates or programmable logic such as software and/or computer program instructions executed by a processor. Other kinds of programmable logic include programmable processors, programmable digital logic (e.g., a field programmable gate array (FPGA), an erasable programmable read only memory (EPROM), an electrically erasable programmable read only memory (EEPROM), an application specific integrated circuit, ASIC, or any other kind of digital logic, software, code, electronic instructions, flash memory, optical disks, CD-ROMs, DVD ROMs, magnetic or optical cards, other types of machine-readable mediums suitable for storing electronic instructions, or any suitable combination thereof. Such data storage media may also provide a data storage means for use in conjunction with the smell deliver system to store any data created.

1-137. (canceled)

138. A smell delivery device comprising:
a delivery channel for receiving a substance from a canister, the substance configured to produce an olfactory output, such as a smell;
an output component through which the substance is emitted; and
one or more airflow generating elements configured to generate airflow to transport the substance from the canister to the output component;
a flow controller for controlling the flow rate, or concentration, of the substance through the delivery channel to the output component;
wherein the flow controller is configured to control the flow rate, or concentration of the substance from the delivery channel to the output component in response to feedback from at least one of:
a) sensor configured to sense the flow rate, or concentration of the substance, through the delivery channel;
b) an environmental sensor configured to sense environmental conditions; and
c) a user feedback device configured to receive an input from a user.

139. The smell delivery device of claim 138, wherein the user feedback device is configured to receive input from the user indicating one or more of:
a) whether the olfactory output is at a level the user can/cannot sense;
b) the emotional response of the user to the olfactory output;
c) the duration, and/or the intensity of the user's perception/sensation of the olfactory output; and
d) a comparison between different olfactory outputs.

140. The smell delivery device of claim 139, wherein, when the user feedback device receives input from the user indicating that the user cannot sense the olfactory output, the flow controller is configured to modulate the flow rate.

141. The smell delivery device of claim 138, wherein the environmental sensor is configured to sense at least one of:
a) the temperature of the environment,
b) the humidity of the environment,
c) the pressure of the environment, and
d) the amount of/identity of pollutants present in the environment.

142. The smell delivery device of claim 141, wherein the flow controller is configured to modulate the flow rate of the substance, in the event that one of:
a) the humidity of the environment or at a location within the smell delivery device is above a predefined threshold;
b) the temperature of the environment or at a location within the smell delivery device is above a predefined threshold; or
c) the pressure of the environment or at a location within the smell delivery device is above a predefined threshold.

143. The smell delivery device of claim 141, wherein, if the amount of pollutants is above a certain threshold, the smell delivery device is configured to stops the flow of the substance.

144. The smell delivery device of claim 138, wherein the sensor configured to sense the flow rate, or concentration, of the substance is configured to communicate the flow rate, or concentration, measurement to the flow controller, and, when the measured flow rate, or concentration, is different than the intended flow rate, the flow controller is configured to modulate the flow rate until the intended flow rate is reached and stabilised.

145. (canceled)

146. The smell delivery device of claim 138, further comprising a distance sensor to determine the distance of the output component to the location of the user, and wherein as the determined distance increases, the flow controller is configured to modulate the flow rate.

147. The smell delivery device of claim 138, further comprising:
a second delivery channel for receiving a second substance from a second canister, the second substance configure to produce a second olfactory output, or alter the olfactory output associated with the first substance;
a second output component through which the second substance is emitted; and
wherein the flow controller is configured to control the flow rate, or concentration, of the second substance from the second delivery channel to the second output component in response to feedback from at least one of:
a) a second sensor positioned in the second delivery channel configured to sense the flow rate, or concentration, of the second substance through the second delivery channel;
b) the environmental sensor configured to sense environmental conditions; and
c) the user feedback device configured to receive an input from a user.

148-155. (canceled)

156. A method of delivering smell to a user, the method comprising:
receiving instructions to emit a flow of a first substance, wherein the substance has an olfactory output, such as a smell, associated therewith;
beginning the flow of the first substance at a first flow rate, or concentration;
receiving feedback from one or more of:
a) a sensor configured to sense the flow rate, or concentration of the substance, through the delivery channel;
b) an environmental sensor configured to sense environmental conditions; and
c) a user feedback device configured to receive an input from a user; and
in response to the feedback: changing the flow rate, or concentration, of the first substance.

157. The method of claim 156, wherein the feedback is from the user feedback device and comprises an indication of at least one of:
a) whether the user can sense the olfactory output;
b) the duration of the user's perception of the olfactory output; and
c) the user's emotional response to the olfactory output.

158. The method of claim 156, wherein the feedback is from the environmental sensor and comprises at least one of:
a) an indication of whether the humidity of the environment or at a location within the smell delivery device is above a predefined threshold;
b) an indication of whether the temperature of the environment or at a location within the smell delivery device is above a predefined threshold; and
c) an indication of whether the pressure of the environment or at a location within the smell delivery device is above a predefined threshold.

159. The method of claim 156, wherein the feedback is from the sensor configured to sense the flow rate, or concentration, of the substance, through the delivery channel, and wherein the feedback comprises an indication that the measured flow rate is different to the intended flow rate, and in response, modulating the flow rate until the intended flow rate is reached and stabilised.

160-162. (canceled)

163. A smell sensing system, comprising:
a smell delivery device for delivering an olfactory output, comprising:
a delivery channel for receiving a substance from a canister, the substance configured to produce an olfactory output;
an output component through which the substance is emitted; and
one or more airflow generating elements configured to generate airflow to transport the substance from the canister to the output component; and
a smell sensing device for detecting the olfactory output delivered by the smell delivery device, comprising:
at least one gas sensor configured to, in response to the olfactory output, generate sensor information corresponding to the olfactory output;
wherein the smell sensing device is configured to output the sensor information to a processor.

164. The smell sensing system of claim 163, wherein the sensor information comprises an indication of one or more of:
a) presence of the olfactory output;
b) an intensity of the olfactory output;
c) an identification of the smell or a type of smell associated with the olfactory output;
d) a pulse duration of the olfactory output;
e) a duration between subsequent pulses of the olfactory output;
f) a base line of the olfactory output; and
g) whether the olfactory output is static or dynamic.

165. The smell sensing system of claim 163, wherein the smell delivery device is configured to output delivery information to the processor corresponding to the substance emitted.

166. The smell sensing system of claim 165, wherein the delivery information is indicative of one or more of:
a) the flow rate or concentration of the substance;
b) pressure of a pump of the airflow generating elements of the smell delivery device;
c) selection of delivery channels of the smell delivery device;
d) selection or degree of opening of valves of the delivery channel of the smell delivery device;
e) an identification of the smell or a type of smell;
f) a pulse duration of the smell;
g) a duration between subsequent pulses of the smell;
h) a smell base line; and
i) whether the smell is static or dynamic.

167. The smell sensing system of claim 163, wherein the smell delivery device is configured to receive instructions from the processor, and wherein the smell delivery device is configured to, in response to receiving the instructions, adjust the delivery of the olfactory output.

168. The smell sensing system of claim 165, further comprising the processor, and wherein the processor is configured to correlate the sensor information with the delivery information to determine a validation of the delivery of the smell by the smell delivery device.

169. The smell sensing system of claim 163, wherein the smell delivery device comprises:
a flow controller for controlling the flow rate, or concentration, of the substance through the delivery channel to the output component;
wherein the flow controller is configured to control the flow rate, or concentration of the substance from the delivery channel to the output component in response to feedback from at least one of:
a) a sensor configured to sense the flow rate, or concentration of the substance, through the delivery channel;
b) an environmental sensor configured to sense environmental conditions; and
c) a user feedback device configured to receive an input from a user.
